(19) United States
(12) Patent Application Publication
(10) Pub. No.: US 2018/0161310 A1
(43) Pub. Date: Jun. 14, 2018

(54) COMPOUNDS AND METHODS FOR THE TREATMENT OF NEUROLOGICAL DISORDERS

(71) Applicant: Teleos Therapeutics, LLC, Concord, MA (US)

(72) Inventors: Randall T. Peterson, Salt Lake City, UT (US); David Kokel, Oakland, CA (US); Tama Evron, Lexington, MA (US); Andrea Velenich, Wilmington, MA (US)

(21) Appl. No.: 15/835,027

(22) Filed: Dec. 7, 2017

Related U.S. Application Data
(60) Provisional application No. 62/431,539, filed on Dec. 8, 2016.

(51) Int. Cl. A61K 31/415; A61K 31/5377; A61K 31/341; A61P 25/08

(52) U.S. Cl. CPC A61K 31/415; A61P 25/08; A61K 31/341; A61K 31/5377

(57) ABSTRACT

Compounds that inhibit synaptic hyperexcitability, pharmaceutical compositions of these compounds, and methods of using the compounds and pharmaceutical compositions thereof to treat diseases and/or disorders such as epilepsy, neuropathic pain, spinal cord injury spasticity, morphine-induced hyperalgesia, schizophrenia, and autism spectrum disorders are disclosed. In some embodiments, the compounds have the general structure of Formula 1 or Formula 2:

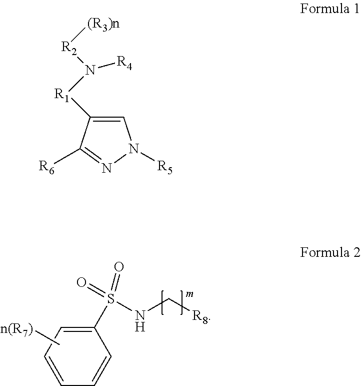

### BACKGROUND

The epilepsies are chronic neurological disorders characterized by recurrent seizures. Approximately 50 million people worldwide have epilepsy, making it one of the most common neurological diseases. Globally, an estimated 2.4 million people are diagnosed with epilepsy each year. Idiopathic epilepsy the is most common type of epilepsy, affecting about 60% of people with the disorder, and has no identifiable cause. Epilepsies with a known cause are called secondary epilepsies, and can be caused by factors including brain damage from prenatal or perinatal injuries, congenital abnormalities or genetic conditions associated with brain malformations, severe head injury, stroke, infections such as meningitis, encephalitis, and neurocysticercosis, certain genetic disorders, and brain tumors.

Traditional antiepileptic drugs such as carbamazepine, clonazepam, ethosuximide, phenobarbitone, phenytoin, phenyloin, valproate, and certain other benzodiazepines remain widely prescribed, but suffer from a range of side effects. Newer drugs, such as felbamate, gabapentin, lamotrigine, levetiracetam, oxcarbazepine, tiagabine, topimarate, vigabartrin, and zonisamide have been developed, and they do show some improved efficacies and side effect profiles compared to traditional antiepileptics. However, there is a significant group of patients that are resistant to currently available therapeutics.

Hyperpolarizing synaptic inhibition via aminobutyric acid type A receptors (GABA_{A}R) and glycine receptors (GlyR) is modulated by the intracellular concentration of Cl⁻ in postsynaptic neurons. The low Cl⁻ concentration required for this inhibition is established and maintained in the mature brain by the potassium chloride cotransporter KCC2, encoded by the SLC12A5 gene. KCC2 extrudes Cl⁻ primarily from CNS neurons, stabilizing inhibitory neurotransmission under normal conditions. Consistent with its importance to synaptic inhibition, KCC2 deficiencies have been linked to epilepsy in rodents and human patients. KCC2 variants have been identified in families with fever-induced and generalized epilepsies as well as severe infantile-onset epilepsy. Moreover, pharmacological inhibition of KCC2 has been shown to induce epileptiform discharges in hippocampal slices and in mouse hippocampus.

KCC2 loss of function has also been reported in other neurological disorders that involve impaired GABA_{A} inhibition and synaptic hyperexcitability, including neuropathic pain, spinal cord injury spasticity, morphine-induced hyperalgesia, schizophrenia, and autism spectrum disorders.

### SUMMARY

In a first aspect, described herein is a pharmaceutical composition comprising a compound of Formula 1:

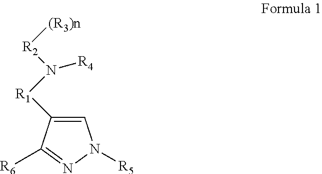

or a pharmaceutically acceptable salt, hydrate, solvate, or N-oxide thereof, wherein:
R₁ is selected from carbonyl and sulfonyl;
R₂ is selected from phenyl, pyridine, and thiazole;
n is 0, 1, 2, 3, or 4;
each R₃ is independently selected from H, halogen, C₁-C₄ alkyl, C₁-C₄ alkoxy, C₁-C₄haloalkyl, amide, acetamide, sulfonamide, sulfone, nitro, nitrile, and alkane nitrate;
R₄ is selected from H and C₁-C₄ alkyl;
R₅ is C₁-C₆ alkyl; and
R₆ is C₁-C₆ alkoxy;
and a pharmaceutically acceptable vehicle.

In some embodiments, each R₃ is independently selected from methyl, methoxy, trifluoromethyl, fluoride, and chloride.

In some embodiments, R₄ is selected from H and methyl.

In some embodiments, n is 1.

In some embodiments, R₅ is propyl.

In some embodiments, R₆ is propoxy.

In some embodiments, the compound of Formula 1 is selected from:

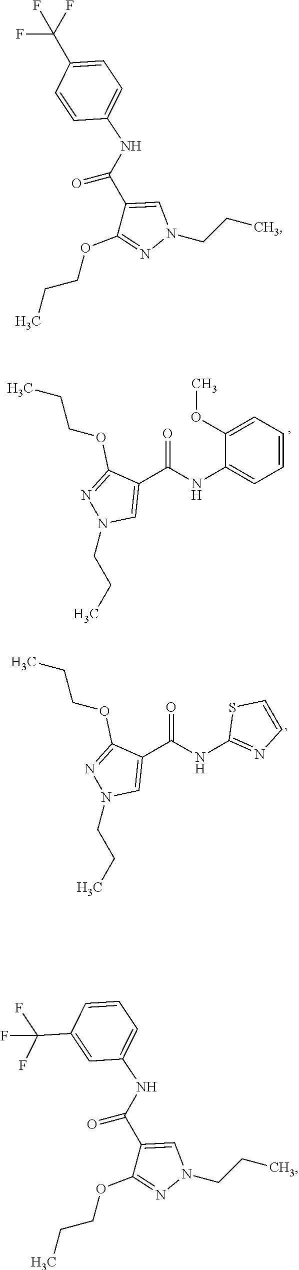 [SMILES: CCCOc1nn(CCC)cc1C(=O)Nc1ccc(C(F)(F)F)cc1 ; CCCOc1nn(CCC)cc1C(=O)Nc1ccccc1OC ; CCCOc1nn(CCC)cc1C(=O)Nc1nccs1 ; CCCOc1nn(CCC)cc1C(=O)Nc1cccc(C(F)(F)F)c1]

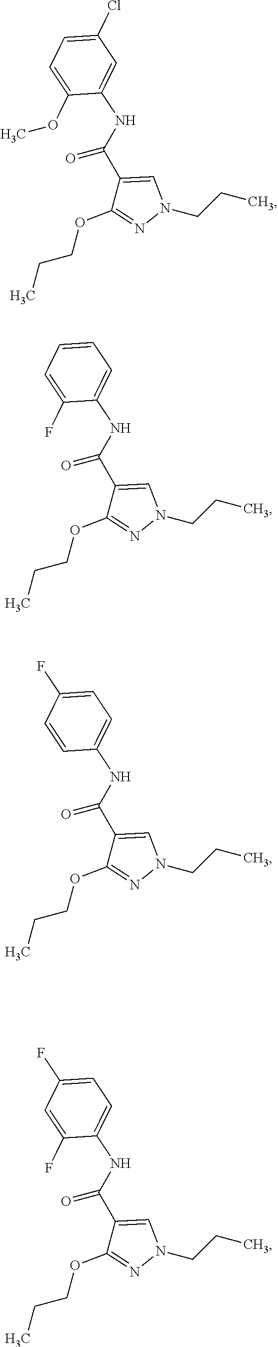 [SMILES: CCCOc1nn(CCC)cc1C(=O)Nc1cc(Cl)ccc1OC ; CCCOc1nn(CCC)cc1C(=O)Nc1ccccc1F ; CCCOc1nn(CCC)cc1C(=O)Nc1ccc(F)cc1 ; CCCOc1nn(CCC)cc1C(=O)Nc1ccc(F)cc1F]

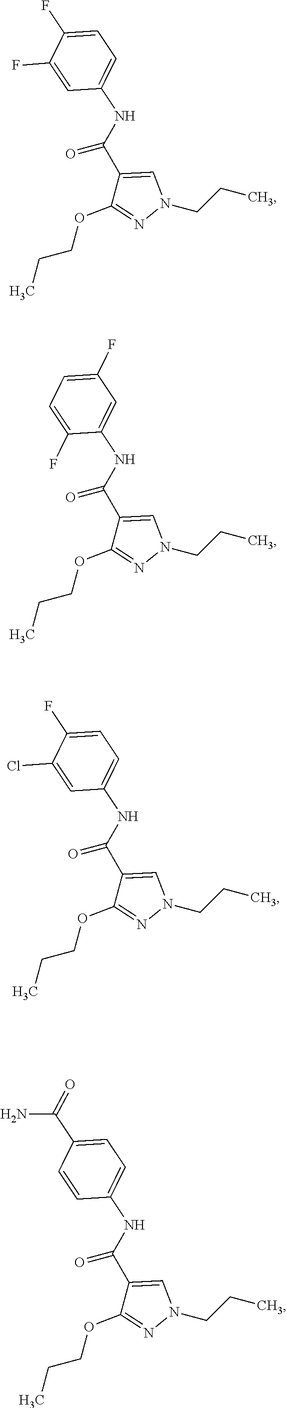 [SMILES: CCCOc1nn(CCC)cc1C(=O)Nc1ccc(F)c(F)c1 ; CCCOc1nn(CCC)cc1C(=O)Nc1cc(F)ccc1F ; CCCOc1nn(CCC)cc1C(=O)Nc1ccc(F)c(Cl)c1 ; CCCOc1nn(CCC)cc1C(=O)Nc1ccc(C(N)=O)cc1]

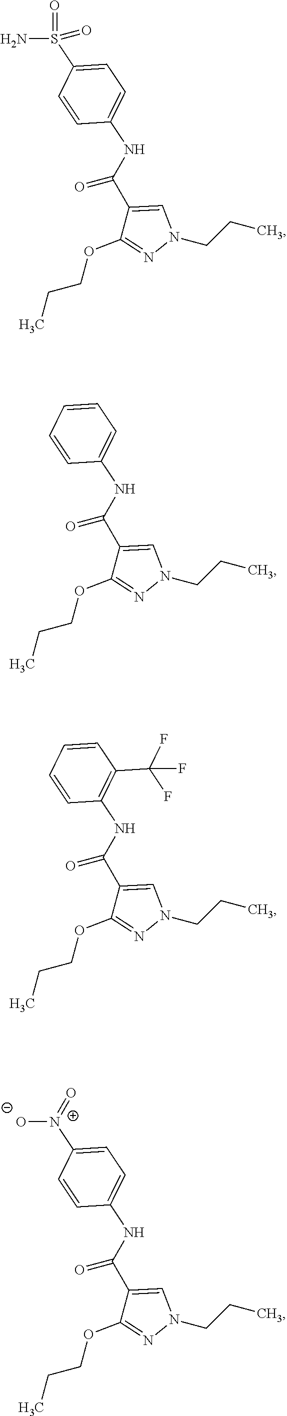 [SMILES: CCCOc1nn(CCC)cc1C(=O)Nc1ccc(S(N)(=O)=O)cc1 ; CCCOc1nn(CCC)cc1C(=O)Nc1ccccc1 ; CCCOc1nn(CCC)cc1C(=O)Nc1ccccc1C(F)(F)F ; CCCOc1nn(CCC)cc1C(=O)Nc1ccc([N+](=O)[O-])cc1]

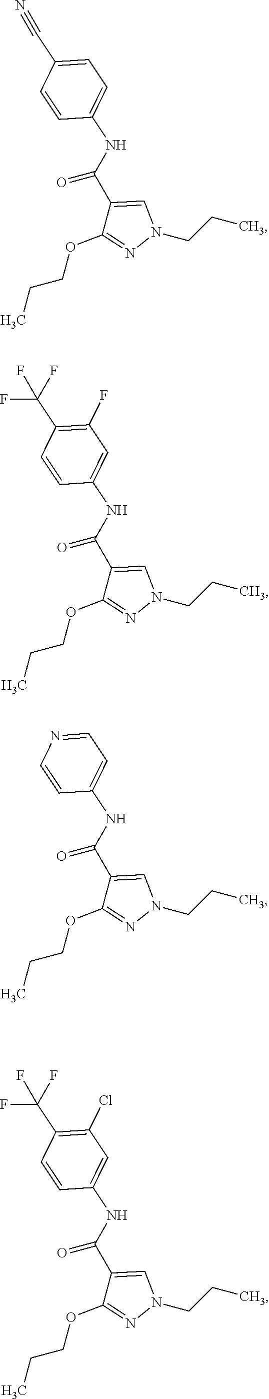 [SMILES: CCCOc1nn(CCC)cc1C(=O)Nc1ccc(C#N)cc1 ; CCCOc1nn(CCC)cc1C(=O)Nc1ccc(C(F)(F)F)c(F)c1 ; CCCOc1nn(CCC)cc1C(=O)Nc1ccncc1 ; CCCOc1nn(CCC)cc1C(=O)Nc1ccc(C(F)(F)F)c(Cl)c1]

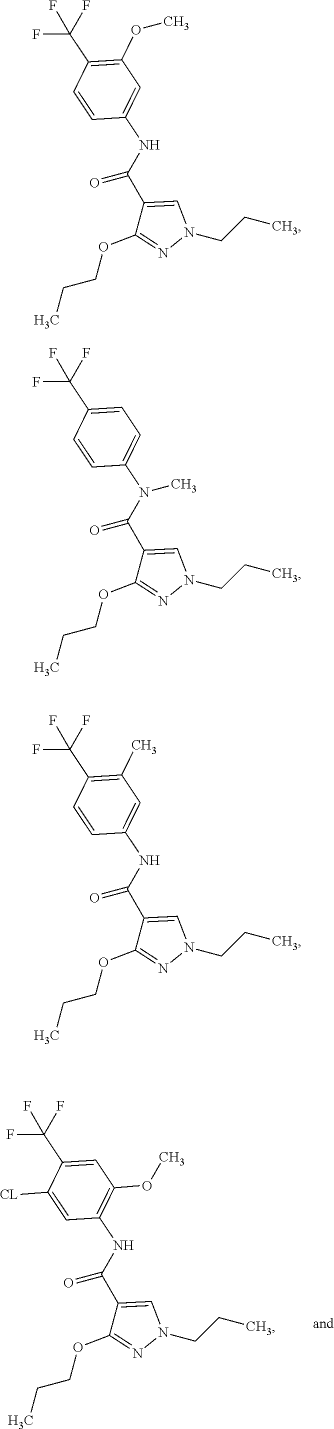

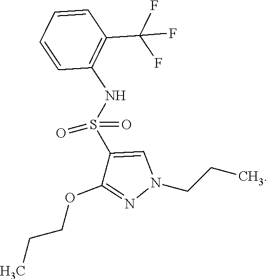 [SMILES: CCCOc1nn(CCC)cc1S(=O)(=O)Nc1ccccc1C(F)(F)F]

In a second aspect, described herein is a pharmaceutical composition comprising a compound of Formula 2:

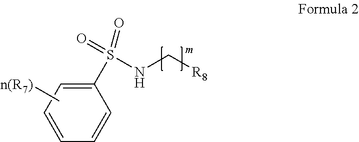

or a pharmaceutically acceptable salt, hydrate, solvate, or N-oxide thereof, wherein:
n is 1, 2, 3, or 4;
each R₇ is independently selected from C₁-C₄ alkyl, C₁-C₄ alkoxy, and halogen;
m is 1, 2, 3, or 4; and
R₈ is selected from tetrahydrofuran and a morpholine moiety;
and a pharmaceutically acceptable vehicle.

In some embodiments, n is selected from 2 and 3.

In some embodiments, each R₇ is independently selected from methyl, methoxy, isopropyl, and bromide.

In some embodiments, m is selected from 1 and 2.

In some embodiments, the compound of Formula 2 is selected from:

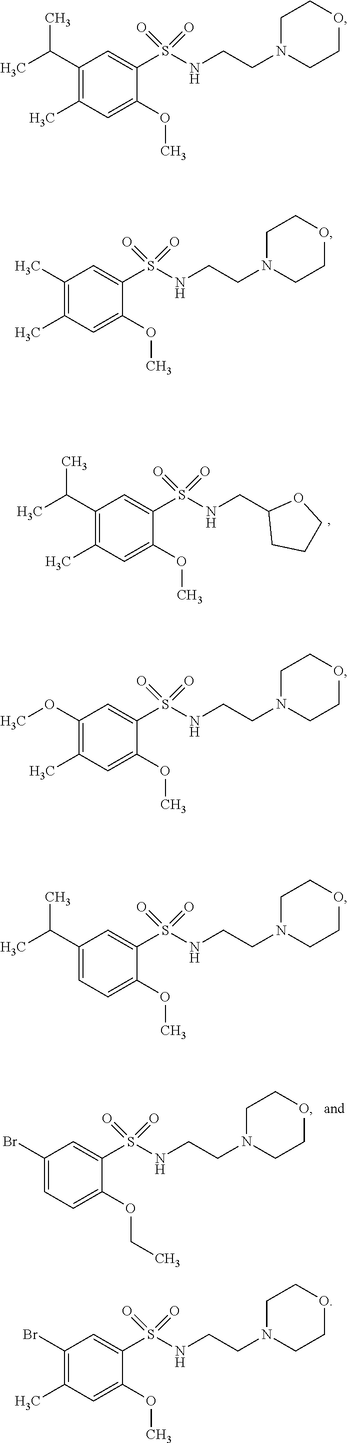

In a third aspect, a pharmaceutical composition of the first or second aspect is formulated for oral administration.

In some embodiments, the pharmaceutical composition comprises 2.5 mg to 1000 mg of the compound of Formula 1 and/or of Formula 2.

In some embodiments, the pharmaceutical composition comprises an amount of the compound of Formula 1 and/or of Formula 2 selected from 2.5 mg, 5 mg, 10 mg, 25 mg, 50 mg, 100 mg, 250 mg, 500 mg, and 1000 mg.

In a fourth aspect, methods for treating a disease and/or condition characterized by synaptic hyperexcitability are provided.

In some embodiments, the methods comprise administering a pharmaceutical compound of the first or second aspects, or a combination thereof, to a patient.

In some embodiments, the disease and/or condition is selected from epilepsy, neuropathic pain, spinal cord injury spasticity, morphine-induced hyperalgesia, schizophrenia, and autism spectrum disorders.

In some embodiments, the pharmaceutical compounds provided by the present disclosure are administered to provide a dose of 5 mg to 2.5 g per day of the compound of Formula 1 and/or Formula 2.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D are representative images of zebrafish behavioral profiles comprised of a battery of individual behavioral assays labeled ASR1, AVSR1, ASR2, VSR1, VSR2, VSR3, VSR4, AVSR2, VSR5, AVSR3, and VSR6. The activity of the zebrafish in the individual behavioral assays combine to form a behavioral profile in which a unit-less measure of zebrafish motion, the Motion Index (MI), is plotted over time and presents a quantification of the motor activity of at least about 8 zebrafish larvae in each well of a 96-well plate during the battery of behavioral assays. Zebrafish larvae were treated as follows: DMSO (control) or VU0463271 (FIG. 1A); DMSO (control) or pentylentetrazol (PTZ; FIG. 1B); DMSO (control), VU0463271 or VU0463271+Diazepam (FIG. 1C); DMSO (control), VU0463271 or VU0463271+Tiagabine (FIG. 1D).

FIGS. 2A-2C are images of representative behavioral profiles (average MI) (FIGS. 2A and 2B) and line graphs (FIG. 2C) demonstrating the ability of various compounds to suppress or “rescue” the hyperexcitability phenotype produced by exposing zebrafish larvae to VU0463271. FIG. 2A is a representative image demonstrating the ability of tiagabine acid to suppress the VU0463271-induced phenotype (behavioral assay AVSR3). FIG. 2B is a representative image demonstrating the ability of valproic acid to suppress the VU0463271-induced phenotype (note in particular the activity during behavioral assays AVSR1 and AVSR3). FIG. 2C includes graphical representations of the phenotypic rescue of the VU0463271-induced phenotype by the indicated anti-seizure drug at various concentrations.

FIGS. 3A-3C are images of representative scatter plots (FIGS. 3A and 3B) and histograms (FIG. 3C) demonstrating the ability of various compounds to suppress or rescue the VU0463271-induced phenotype. FIGS. 3A and 3B are representative small molecule library-wide 2D scatter plots illustrating the suppression of the VU0463271-induced phenotype by various “hits” in the small molecule library. The data shown in FIG. 3A were filtered for overall activity, and the data shown in FIG. 3B were filtered for compounds with scores <0, compounds having distances from DMSO<0.4, and compounds with mean MI>3. FIG. 3C is a histogram representing the distribution of screen scores for about 240 compounds that were selected from the primary screen for their ability to suppress the VU0463271-induced phenotype and retested in triplicate.

FIGS. 4A-4C are images of representative behavioral profiles (average MI) of the top three hit compounds identified in primary screens, along with their corresponding chemical structures. TT0023831 (FIG. 4A) and TT0042607 (FIG. 4B) were identified in a VU0463271 suppressor screen, while TT0011235 was identified in a VU0240551 suppressor screen (FIG. 4C).

FIGS. 5A and 5B are images of representative line graphs (FIG. 5A) and scatter plots (FIG. 5B) demonstrating the ability of three hit compounds to suppress VU0463271 or VU0240551-induced phenotypes. FIG. 5A includes representative line graphs in which each compound was tested at the various concentrations indicated and plotted against their respective rescue score. FIG. 5B includes representative 2D scatter plots in which each compound was analyzed for their respective plot distance from the VU0463271 or VU0240551-induced projections and towards the controls (DMSO). The ability of the compound tested to rescue the KCC2 inhibited phenotype can be seen in the shift in the phenotype of compound-treated animals (red dots) away from KCC2-inhibited phenotype (purple dots) and toward the wild-type, DMSO-treated phenotype (green dots).

FIGS. 6A-6C are images of representative behavioral profiles (average MI) of TT0023831 (FIG. 6A), TT0042607 (FIG. 6B), and TT0011235 (FIG. 6C), and their respective ability to suppress (or not suppress) the PTZ-induced phenotype.

FIGS. 7A and 7B are images of representative behavioral profiles (average MI) of TT0023831 (FIG. 7A) and TT0042607 (FIG. 7B), and their ability to suppress the VU0463271-induced phenotype at the concentrations indicated.

FIG. 8 includes representative dose response curves for TT0023831 and TT0042607, based on their respective suppression scores (combined measurement of VU0463271 suppression, plot distance from DMSO controls, and sedation level) at the concentrations indicated.

FIG. 9 includes representative IV curves (current-voltage curves) characterizing the intrinsic excitability of TT0023831 and TT0042607 in gramicidin perforated patch clamp studies.

FIG. 10 includes representative dose response curves for a structural derivative of TT0042607, based on its respective suppression scores (combined measurement of VU0463271 suppression, plot distance from DMSO controls, and sedation level) at the concentrations indicated.

FIG. 11 depicts graphs indicating the degree of suppression of the KCC2-inhibited phenotype by the indicated compounds, where a score of 1 represents complete restoration of KCC2-treated zebrafish to normal.

FIGS. 12A-12C depict graphs indicating the degree of suppression of the KCC2-inhibited phenotype, where a score of 1 represents complete restoration of KCC2-treated zebrafish to normal, and the 10-point concentration-response curve (CRC) as determined by CHO-Kv7.2/7.3 (KCNQ2/3) ⁸⁶Rb efflux assay for TT0000352 (FIG. 12A), TT0000369 (FIG. 12B), and TT0000372 (FIG. 12C).

FIGS. 13A-13C depict 10-point concentration-response curve (CRC) as determined by sodium channel FLIPR assays for tetracaine (FIG. 13A), tetrodotoxin (TTX) (FIG. 13B), and TT0023831 (FIG. 13C).

### DETAILED DESCRIPTION

In the following sections, various exemplary compositions and methods are described in order to detail various embodiments. It will be evident to one skilled in the art that practicing the various embodiments does not require the employment of all of the specific details outlined herein, but rather that formulation composition, dosing, administration routes and other specific details may be modified through experimentation. In some embodiments, well known methods or components have not been included in the description.

### Definitions

“Alkyl” by itself or as part of another substituent refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclo-propan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In various embodiments, the desired number of carbon atoms in an alkyl group is described, for example “C₁-C₄ alkyl” refers to an alkyl group having from 1 to 4 carbon atoms. In some embodiments, an alkyl group comprises from 1 to 20 carbon atoms. In other embodiments, an alkyl group comprises from 1 to 10 carbon atoms. In still other embodiments, an alkyl group comprises from 1 to 8 carbon atoms. In other embodiments, an alkyl group comprises from 1 to 6 carbon atoms. In other embodiments, an alkyl group comprises from 1 to 4 carbon atoms.

“Aryl” by itself or as part of another substituent refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryl group comprises from 6 to 20 carbon atoms. In other embodiments, and aryl group comprises from 6 to 12 carbon atoms.

“Compounds” refers to compounds encompassed by the generic formulae disclosed herein and includes any specific compounds within those formulae whose structure is disclosed herein. Compounds may be identified either by their chemical structure and/or chemical name. If any chemical structure and chemical name are found to conflict, the chemical structure is determinative of the identity of the compound. Compounds disclosed herein may contain one or more chiral centers and/or double bonds and therefore, may exist as stereoisomers, such as double-bond isomers (i.e., geometric isomers), enantiomers or diastereomers. Accordingly, when stereochemistry at chiral centers is not specified, the chemical structures depicted herein encompass all possible configurations at those chiral centers including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. Compounds disclosed herein may also exist in several tautomeric forms including the enol form, the keto form and mixtures thereof. Accordingly, the chemical structures depicted herein encompass all possible tautomeric forms of the illustrated compounds.

Compounds disclosed herein also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁷O and ¹⁸O. Compounds disclosed herein may exist in unsolvated forms as well as solvated forms, including hydrated forms and as N-oxides. Certain compounds disclosed herein may exist in multiple crystalline or amorphous forms. In general, all physical forms are equivalent for the uses contemplated herein and are intended to be within the scope of the present disclosure. Further, it should be understood, when partial structures of compounds are illustrated, an asterisk (*) indicates the point of attachment of the partial structure to the rest of the molecule.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In some embodiments, the cycloalkyl group is (C₃-C₁₀) cycloalkyl. In certain embodiments, the cycloalkyl group is (C₃-C₇) cycloalkyl.

“Cycloheteroalkyl” by itself or as part of another substituent refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, and Si. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, tetrahydrofuran, piperazine, piperidine, pyrazolidine, pyrrolidine, quinuclidine, and the like.

“Heteroalkyl” by itself or as part of another substituent refers to alkyl groups in which one or more of the carbon atoms (and any associated hydrogen atoms) are independently replaced with the same or different heteroatomic groups. Typical hetero-atomic groups which can be included in these groups include, but are not limited to, —O—, —S—, —O—O—, —S—S—, —O—S—, —NR³⁴R³⁵—, ══N—N══, —N══N—, —N══N—NR³⁶R³⁷, —PR³⁸—, —P(O)₂—, —POR³⁹—, —O—P(O)₂—, —SO—, —SO₂—, SnR⁴⁰R⁴¹— and the like, where R³⁴, R³⁵, R³⁶, R³⁷, R³⁸R³⁹, R⁴¹ and R⁴¹ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group is a 5-20 membered heteroaryl. In other embodiments, the heteroaryl group is a 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Pharmaceutical composition” refers to at least one compound disclosed herein and a pharmaceutically acceptable vehicle, with which the compound is administered to a patient.

“Patient” includes mammals, such as for example, humans.

“Prodrug” refers to a derivative of a drug molecule that requires a transformation within the body to release the active drug. Prodrugs are frequently, although not necessarily, pharmacologically inactive until converted to the parent drug. A hydroxyl-containing drug may be converted, for example, to an ester, carbonate, acyloxyalkyl or a sulfonate prodrug, which may be hydrolyzed in vivo to provide the hydroxyl compound. Prodrugs for drugs with functional groups different than those listed above are well known to the skilled artisan.

“Promoiety” refers to a form of protecting group that when used to mask a functional group within a drug molecule converts the drug into a prodrug. Typically, the pro-moiety will be attached to the drug via one or more bonds that are cleaved by enzymatic or non-enzymatic means in vivo.

“Therapeutically effective amount” means the amount of a compound that, when administered to a patient for treating a disease or disorder, is sufficient to treat the disease or disorder. The “therapeutically effective amount” will vary depending on the compound, the disease or disorder and its severity and the age, weight, etc., of the patient to be treated. In some embodiments, “therapeutically effective amount” refers to an amount of a prodrug that, when administered to a patient for treating a disease or disorder, is sufficient to result in an in vivo concentration of the active drug in the patient sufficient to treat the disease or disorder. In such embodiments, the “therapeutically effective amount” takes into account the rate and efficiency at which the prodrug is metabolized into the active drug. In other embodiments, “therapeutically effective amount” refers to an amount of the active drug that, following prodrug metabolization, is sufficient to effect treatment of a disease or disorder in a patient. It will be evident to those of skill in the art whether a dosage provided herein refers to the prodrug amount or to the resulting amount of the active drug.

Reference will now be made in detail to various embodiments. It will be understood that the following description is not intended to limit the present disclosure to those embodiments. To the contrary, it is intended to cover alternatives, modifications, and equivalents as may be included within the spirit and scope of the disclosed subject matter as defined by the appended claims.

### Compounds

The compounds disclosed herein can be used to treat neurological disorders characterized by synaptic hyperexcitability. In some embodiments, neurological disorders characterized by synaptic hyperexcitiability include, but are not limited to, epilepsies, neuropathic pain, spinal cord injury spasticity, morphine-induced hyperalgesia, schizophrenia, and autism spectrum disorders.

In some embodiments, compounds of structural Formula 1 are provided:

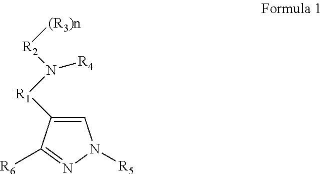

or pharmaceutically acceptable salts, hydrates, solvates, or N-oxides thereof, wherein:
R₁ is selected from carbonyl and sulfonyl;
R₂ is selected from phenyl, pyridine, and thiazole;
n is 0, 1, 2, 3, or 4;
each R₃ is independently selected from H, halogen, C₁-C₄ alkyl, C₁-C₄ alkoxy, C₁-C₄ haloalkyl, amide, acetamide, sulfonamide, sulfone, nitro, nitrile, and alkane nitrate;
R₄ is selected from H and C₁-C₄ alkyl;
R₅ is a C₁-C₆ alkyl; and
R₆ is a C₁-C₆ alkoxy.

In some embodiments, each R₃ is independently selected from methyl, methoxy, trifluoromethyl, and chloride.

In some embodiments, R₄ is selected from hydrogen and methyl.

In some embodiments, n is 1.

In some embodiments, R₅ is propyl.

In some embodiments, R₆ is propoxy.

In one embodiment, R₁ is carbonyl, R₂ is phenyl, n is 1, R₃ is trifluoromethyl, R₄ is H, R₅ is propyl, and R₆ is propoxy.

In one embodiment, R₁ is carbonyl, R₂ is phenyl, n is 1, R₃ is methoxy, R₄ is H, R₅ is propyl, and R₆ is propoxy.

In one embodiment, R₁ is carbonyl, R₂ is thiazole, n is 0, R₄ is H, R₅ is propyl, and R₆ is propoxy.

In one embodiment, R₁ is carbonyl, R₂ is phenyl, n is 2, the R₃ groups are methoxy and chloride, R₄ is H, R₅ is propyl, and R₆ is propoxy.

In one embodiment, R₁ is carbonyl, R₂ is phenyl, n is 1, R₃ is fluoride, R₄ is H, R₅ is propyl, and R₆ is propoxy.

In one embodiment, R₁ is carbonyl, R₂ is phenyl, n is 2, the R₃ groups are both fluoride, R₄ is H, R₅ is propyl, and R₆ is propoxy.

In one embodiment, R₁ is carbonyl, R₂ is phenyl, n is 2, the R₃ groups are fluoride and chloride, R₄ is H, R₅ is propyl, and R₆ is propoxy.

In one embodiment, R₁ is carbonyl, R₂ is phenyl, n is 1, R₃ is an amide, R₄ is H, R₅ is propyl, and R₆ is propoxy.

In one embodiment, R₁ is carbonyl, R₂ is phenyl, n is 1, R₃ is a sulfonamide, R₄ is H, R₅ is propyl, and R₆ is propoxy.

In one embodiment, R₁ is carbonyl, R₂ is phenyl, n is 1, R₃ is a nitro group, R₄ is H, R₅ is propyl, and R₆ is propoxy.

In one embodiment, R₁ is carbonyl, R₂ is phenyl, n is 1, R₃ is a nitrile group, R₄ is H and R₅ and R₆ are as defined above for structural Formula I.

In one embodiment, R₁ is carbonyl, R₂ is phenyl, n is 1, R₃ is a nitro group, R₄ is H, R₅ is propyl, and R₆ is propoxy.

In one embodiment, R₁ is carbonyl, R₂ is phenyl, n is 2, the R₃ groups are fluoride and trifluoromethyl, R₄ is H, R₅ is propyl, and R₆ is propoxy.

In one embodiment, R₁ is carbonyl, R₂ is pyridine, n is 0, R₄ is H, R₅ is propyl, and R₆ is propoxy.

In one embodiment, R₁ is carbonyl, R₂ is phenyl, n is 2, the R₃ groups are chloride and trifluoromethyl, R₄ is H, R₅ is propyl, and R₆ is propoxy.

In one embodiment, R₁ is carbonyl, R₂ is phenyl, n is 2, the R₃ groups are methoxy and trifluoromethyl, R₄ is H, R₅ is propyl, and R₆ is propoxy.

In one embodiment, R₁ is carbonyl, R₂ is phenyl, n is 1, R₃ is trifluoromethyl, R₄ is methyl, R₅ is propyl, and R₆ is propoxy.

In one embodiment, R₁ is carbonyl, R₂ is phenyl, n is 3, the R₃ groups are methoxy, trifluoromethyl, and chloride, R₄ is H, R₅ is propyl, and R₆ is propoxy.

In some embodiments, the compound of Formula 1 is selected from the compounds provided in Table 1.

Table 1 presents representative generalized structures of compounds falling within the scope of Formula 1, as contemplated herein.

TABLE 1

| | | IUPAC |
|---|---|---|
| Compound | Structure | Name/Description |
| TT0042607 | 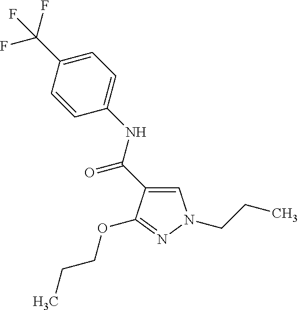 [SMILES: CCCOc1nn(CCC)cc1C(=O)Nc1ccc(C(F)(F)F)cc1] | 3-propoxy-1-propyl- N-(4- (trifluoromethyl)phen- yl)-1H-pyrazole-4- carboxamide |
| TT0042619 | 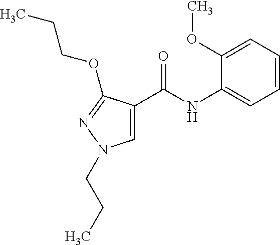 [SMILES: CCCOc1nn(CCC)cc1C(=O)Nc1ccccc1OC] | N-(2- methoxyphenyl)-3- propoxy-1-propyl- 1H-pyrazole-4- carboxamide |
| TT0025322 | 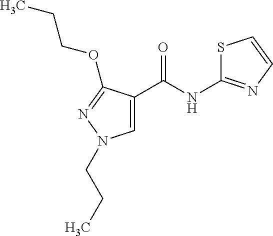 [SMILES: CCCOc1nn(CCC)cc1C(=O)Nc1nccs1] | 3-propoxy-1-propyl- N-(thiazol-2-yl)-1H- pyrazole-4- carboxamide |
| TT0025381 | 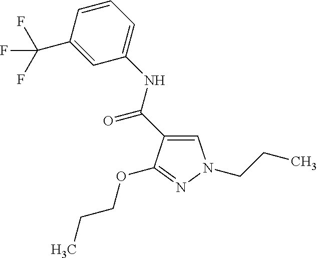 [SMILES: CCCOc1nn(CCC)cc1C(=O)Nc1cccc(C(F)(F)F)c1] | 3-propoxy-1-propyl- N-(3- (trifluoromethyl)phen- yl)-1H-pyrazole-4- carboxamide |
| TT0025358 | 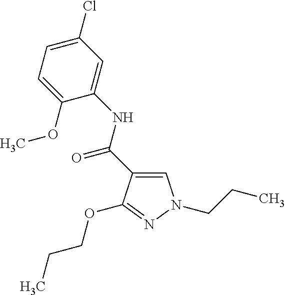 [SMILES: CCCOc1nn(CCC)cc1C(=O)Nc1cc(Cl)ccc1OC] | N-(5-chloro-2- methoxyphenyl)-3- propoxy-1-propyl- 1H-pyrazole-4- carboxamide |
| TT0000347 | 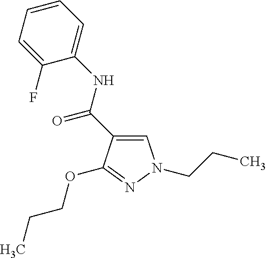 [SMILES: CCCOc1nn(CCC)cc1C(=O)Nc1ccccc1F] | N-(2-fluorophenyl)-3- propoxy-1-propyl- 1H-pyraazole-4- carboxamide |
| TT0000348 | 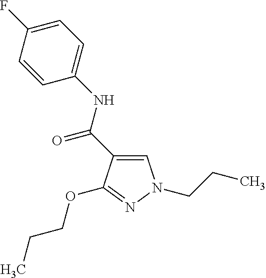 [SMILES: CCCOc1nn(CCC)cc1C(=O)Nc1ccc(F)cc1] | N-(4-fluorophenyl)-3- propoxy-1-propyl- 1H-pyrazole-4- carboxamide |
| TT0000349 | 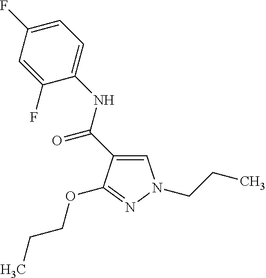 [SMILES: CCCOc1nn(CCC)cc1C(=O)Nc1ccc(F)cc1F] | N-(2,4- difluorophenyl)-3- propoxy-1-propyl- 1H-pyrazole-4- carboxamide |
| TT0000350 | 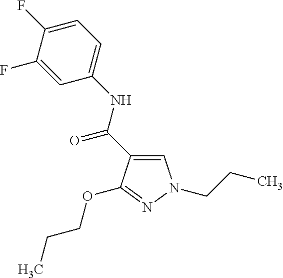 [SMILES: CCCOc1nn(CCC)cc1C(=O)Nc1ccc(F)c(F)c1] | N-(3,4- difluorophenyl)-3- propoxy-1-propyl- 1H-pyrazole-4- carboxamide |
| TT000351 | 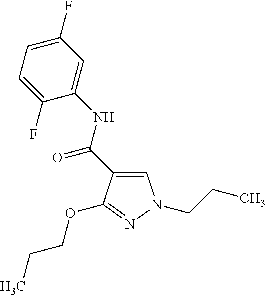 [SMILES: CCCOc1nn(CCC)cc1C(=O)Nc1cc(F)ccc1F] | N-(2,5- difluorophenyl)-3- propoxy-1-propyl- 1H-pyrazole-4- carboxamide |
| TT0000352 | 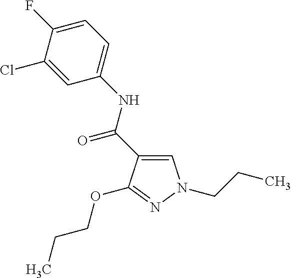 [SMILES: CCCOc1nn(CCC)cc1C(=O)Nc1ccc(F)c(Cl)c1] | N-(3-chloro-4- fluorophenyl)-3- propoxy-1-propyl- 1H-pyrazole-4- carboxamide |
| TT0000353 | 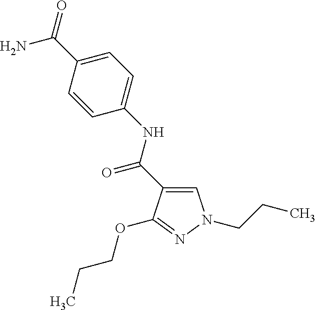 [SMILES: CCCOc1nn(CCC)cc1C(=O)Nc1ccc(C(N)=O)cc1] | N-(4- carbamoylphenyl)-3- propoxy-1-propyl- 1H-pyrazole-4- carboxamide |
| TT0000354 | 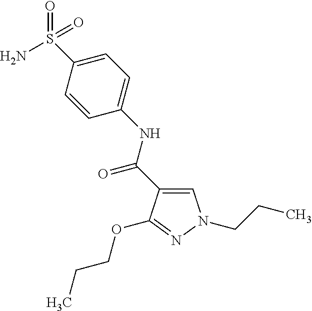 [SMILES: CCCOc1nn(CCC)cc1C(=O)Nc1ccc(S(N)(=O)=O)cc1] | 3-propoxy-1-propyl- N-(4- sulfamoylphenyl)- 1H-pyrazole-4- carboxamide |
| TT0000363 | 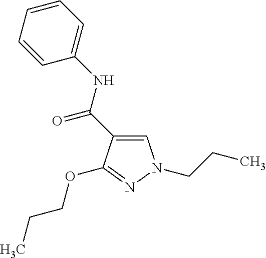 [SMILES: CCCOc1nn(CCC)cc1C(=O)Nc1ccccc1] | N-phenyl-3-propoxy- 1-propyl-1H- pyrazole-4- carboxamide |
| TT0000364 | 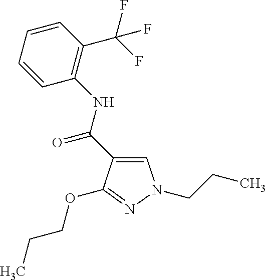 [SMILES: CCCOc1nn(CCC)cc1C(=O)Nc1ccccc1C(F)(F)F] | 3-propoxy-1-propyl- N-(2- (trifluoromethyl)phen- yl)-1H-pyrazole-4- carboxamide |
| TT0000365 | 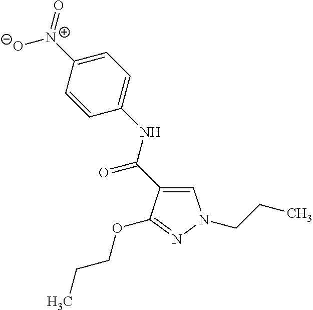 [SMILES: CCCOc1nn(CCC)cc1C(=O)Nc1ccc([N+](=O)[O-])cc1] | N-(4-nitrophenyl)-3- propoxy-1-propyl- 1H-pyrazole-4- carboxamide |
| TT0000366 | 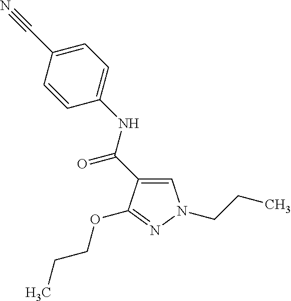 [SMILES: CCCOc1nn(CCC)cc1C(=O)Nc1ccc(C#N)cc1] | N-(4-cyanophenyl)-3- propoxy-1-propyl- 1H-pyrazole-4- carboxamide |
| TT0000367 | 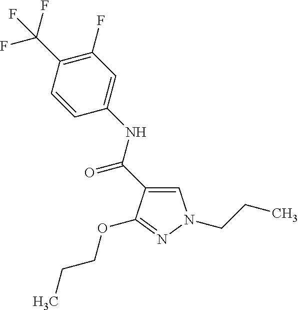 [SMILES: CCCOc1nn(CCC)cc1C(=O)Nc1ccc(C(F)(F)F)c(F)c1] | N-(3-fluoro-4- (trifluoromethyl)phen- yl)-3-propoxy-1- propyl-1H-pyrazole- 4-carboxamide |
| TT0000368 | 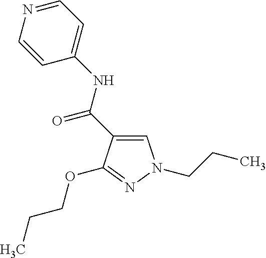 [SMILES: CCCOc1nn(CCC)cc1C(=O)Nc1ccncc1] | 3-propoxy-1-propyl- N-(pyridin-4-yl)-1H- pyrazole-4- carboxamide |
| TT0000369 | 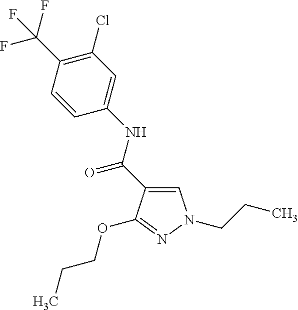 [SMILES: CCCOc1nn(CCC)cc1C(=O)Nc1ccc(C(F)(F)F)c(Cl)c1] | N-(3-chloro-4- (trifluoromethyl)phen- yl)-3-propoxy-1- propyl-1H-pyrazole- 4-carboxamide |
| TT0000370 | 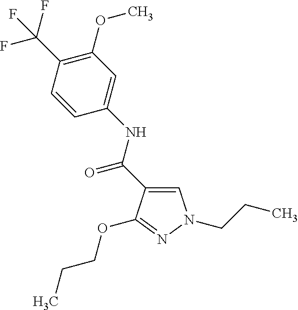 [SMILES: CCCOc1nn(CCC)cc1C(=O)Nc1ccc(C(F)(F)F)c(OC)c1] | N-(3-methoxy-4- (trifluoromethyl)phen- yl)-3-propoxy-1- propyl-1H-pyrazole- 4-carboxamide |
| TT0000371 | 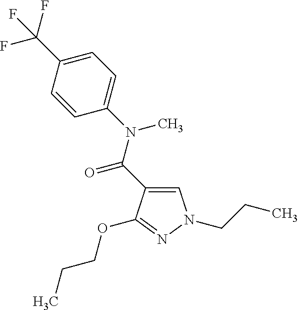 [SMILES: CCCOc1nn(CCC)cc1C(=O)N(C)c1ccc(C(F)(F)F)cc1] | N-methyl-3-propoxy- 1-propyl-N-(4- (trifluoromethyl)phen- yl)-1H-pyrazole-4- carboxamide |
| TT0000372 | 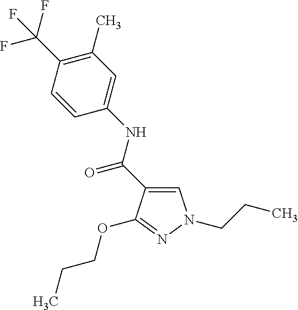 [SMILES: CCCOc1nn(CCC)cc1C(=O)Nc1ccc(C(F)(F)F)c(C)c1] | N-(3-methyl-4- (trifluoromethyl)phen- yl)-3-propoxy-1- propyl-1H-pyrazole- 4-carboxamide |
| TT0000373 | 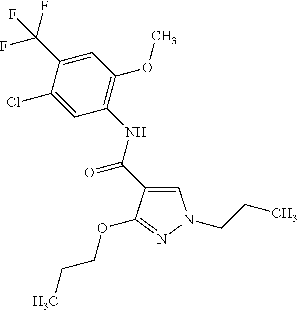 [SMILES: CCCOc1nn(CCC)cc1C(=O)Nc1cc(Cl)c(C(F)(F)F)cc1OC] | N-(5-chloro-2- methoxy-4- (trifluoromethyl)phen- yl)-3-propoxy-1- propyl-1H-pyrazole- 4-carboxamide |
| TT0000374 | 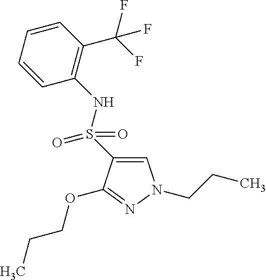 [SMILES: CCCOc1nn(CCC)cc1S(=O)(=O)Nc1ccccc1C(F)(F)F] | 3-propoxy-1-propyl- N-(2- (trifluoromethyl)phen- yl)-1H-pyrazole-4- sulfonamide |

In some embodiments, compounds of structural Formula 2 are provided:

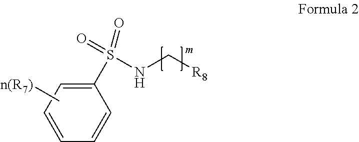

or pharmaceutically acceptable salts, hydrates, solvates, or N-oxides thereof, wherein:
n is 1, 2, 3, or 4;
each R₇ is independently selected from C₁-C₄ alkyl, C₁-C₄ alkoxy, and halogen;
m is 1, 2, 3, or 4; and
R₈ is selected from tetrahydrofuran and a morpholine moiety.

In some embodiments, n is 2. In some embodiments, n is 3.

In some embodiments, each R₇ is independently selected from methyl, methoxy, isopropyl, and bromide.

In some embodiments, m is 1. In some embodiments, m is 2.

In one embodiment, n is 3, the R₇ groups are methyl, isopropyl, and methoxy, m is 2, and R₈ is a morpholine moiety.

In one embodiment, n is 3, the R₇ groups are methyl, methyl and methoxy, m is 2, and R₈ is a morpholine moiety.

In one embodiment, n is 3, the R₇ groups are methyl, isopropyl, and methoxy, m is 1, and R₈ is tetrahydrofuran.

In one embodiment, n is 3, the R₇ groups are methyl, methoxy and methoxy, m is 2, and R₈ is a morpholine moiety.

In one embodiment, n is 2, the R₇ groups are isopropyl and methoxy, m is 2, and R₈ is a morpholine moiety.

In one embodiment, n is 2, the R₇ groups are bromide and methoxy, m is 2, and R₈ is a morpholine moiety.

In one embodiment, n is 3, the R₇ groups are methyl, methoxy, and bromide, m is 2, and R₈ is a morpholine moiety.

In some embodiments, the compound of Formula 2 is selected from the compounds provided in Table 2.

Table 2 presents representative generalized structures of compounds falling within the scope of Formula 1, as contemplated herein.

TABLE 2

| Compound | Structure | IUPAC Name/Description |
|---|---|---|
| TT0023831 | 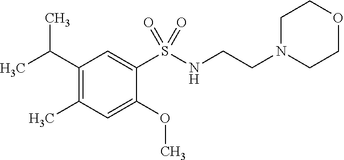 [SMILES: COc1cc(C)c(C(C)C)cc1S(=O)(=O)NCCN1CCOCC1] | 5-isopropyl-2-methoxy-4- methyl-N-(2- morpholinoethyl)benzene- sulfonamide |
| TT0038280 | 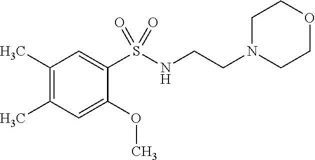 [SMILES: COc1cc(C)c(C)cc1S(=O)(=O)NCCN1CCOCC1] | 2-methoxy-4,5-dimethyl-N- (2- morpholinoethyl)benzene- sulfonamide |
| TT0048274 | 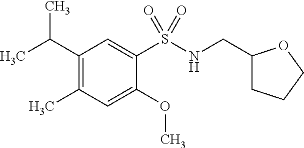 [SMILES: COc1cc(C)c(C(C)C)cc1S(=O)(=O)NCC1CCCO1] | 5-isopropyl-2-methoxy-4- methyl-N-((tetrahydrofuran- 2- yl)methyl)benzenesulfonamide |
| TT0048874 | 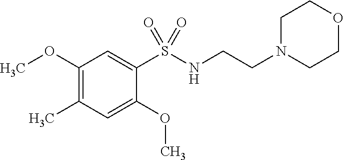 [SMILES: COc1cc(S(=O)(=O)NCCN2CCOCC2)c(OC)cc1C] | 2,5-dimethoxy-4-methyl-N- (2- morpholinoethyl)benzene- sulfonamide |
| TT0048803 | 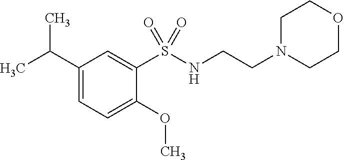 [SMILES: COc1ccc(C(C)C)cc1S(=O)(=O)NCCN1CCOCC1] | 5-isopropyl-2-methoxy-N-(2- morpholinoethyl)benzene- sulfonamide |
| TT0048134 | 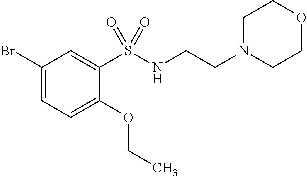 [SMILES: CCOc1ccc(Br)cc1S(=O)(=O)NCCN1CCOCC1] | 5-bromo-2-ethoxy-N-(2- morpholinoethyl)benzene- sulfonamide |
| TT0027052 | 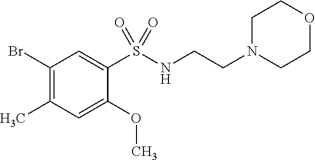 [SMILES: COc1cc(C)c(Br)cc1S(=O)(=O)NCCN1CCOCC1] | 5-bromo-2-methoxy-4- methyl-N-(2- morpholinoethyl)benzene- sulfonamide |

In some embodiments, a compound of structural Formula 3 is provided:

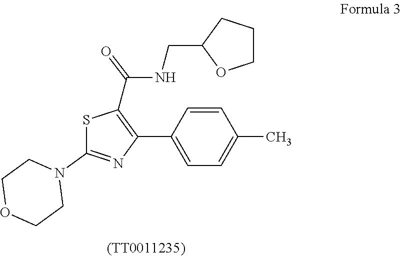 [SMILES: Cc1ccc(-c2nc(N3CCOCC3)sc2C(=O)NCC2CCCO2)cc1]

or pharmaceutically acceptable salts, hydrates, solvates, or N-oxides thereof

Compounds of Formula 1 described herein are partial agonists of the voltage-gated Kv7.2/7.3 (KCNQ2/3) potassium channels. The voltage-gated Kv7 (KCNQ) channels are voltage-dependent potassium channels that are activated at resting membrane potentials, thus providing potent control over neuronal excitability. The antiepileptic drug retigabine activates KCNQ2-5 channels by shifting their voltage-dependent opening to more negative voltages and thereby inhibits synaptic hyperexcitability. However, side effects have limited the clinical use of retigabine. The compounds of Formula 1 present an alternative to retigabine.

Compounds of Formula 2 described herein are sodium channel antagonists. Sodium channels are essential for the initiation and propagation of neuronal firing, and are critical determinants of neuronal excitability. Voltage-gated sodium channels have been show to play a central role in the development and treatment of some epilepsies. Several antiepileptic drugs are sodium channel antagonists, acting on the channels to suppress seizure activity.

### Therapeutic/Prophylactic Uses of the Compounds and Modes of Administration

The compounds described herein can be used to treat and/or prevent synaptic hyperexcitability in patients. In some embodiments, the compounds described herein can be used to treat and/or prevent epileptic seizures, neuropathic pain, spinal cord injury spasticity, morphine-induced hyperalgesia, schizophrenia, and/or autism spectrum disorders. In one embodiment, the compounds described herein can be used to treat and/or prevent epileptic seizures. The present disclosure therefore also provides methods of administering the disclosed compounds to patients in order to achieve a therapeutic benefit, for example treatment or prophylaxis. In some embodiments, the methods comprise administering to a patient a therapeutically effective amount of a compound to treat or prevent synaptic hyperexcitability. In the therapeutic methods disclosed herein, a therapeutically effective amount of the compound is administered to a patient suffering from a condition characterized by synaptic hyperexcitability, including but not limited to epilepsy, neuropathic pain, spinal cord injury spasticity, morphine-induced hyperalgesia, schizophrenia, and autism spectrum disorders. In the prophylactic methods disclosed herein, a therapeutically effective amount of the compound is administered to a patient at risk of developing synaptic hyperexcitability.

In some embodiments, compounds disclosed herein are administered enterally (e.g., oral, rectal, buccal, or sublingual administration) to treat or prevent synaptic hyperexcitability. In other embodiments, compounds disclosed herein are administered parenterally (e.g., via inhalation or injection). In either case, compounds are administered in amounts between about 5 mg to about 2.5 g per day to treat or prevent synaptic hyperexcitability.

Compounds disclosed herein may be used as antiepileptic drugs and can be administered to patients diagnosed with epilepsy. Accordingly, methods for treating epilepsy and/or preventing epileptic seizures are provided. Typically, a therapeutically effective amount of a compound disclosed herein is administered to a patient to treat epilepsy and/or prevent epileptic seizures.

In some embodiments, compounds are administered enterally (e.g., oral, rectal, buccal, or sublingual administration) to treat epilepsy and/or prevent epileptic seizures. However, in other embodiments, compounds are administered parenterally (e.g., via inhalation or injection) to treat epilepsy and/or prevent epileptic seizures. In either case, compounds are administered at a dose of between about 5 mg to about 2.5 g per day to treat epilepsy and/or prevent epileptic seizures.

Compounds may also be used to treat or prevent neuropathic pain, spinal cord injury spasticity, morphine-induced hyperalgesia, schizophrenia, and/or autism spectrum disorders. In some embodiments, a therapeutically effective amount of compound is administered to a patient to treat or prevent any one or more of such diseases or disorders.

In some embodiments, compounds are administered enterally (e.g., oral, rectal, buccal, or sublingual administration) at a dose of about 5 mg to about 2.5 g per day when used to treat or prevent neuropathic pain, spinal cord injury spasticity, morphine-induced hyperalgesia, schizophrenia, and/or autism spectrum disorders. However, in other embodiments, compound may also be administered by inhalation, intravenously or intramuscularly when used to treat or prevent neuropathic pain, spinal cord injury spasticity, morphine-induced hyperalgesia, schizophrenia, and/or autism spectrum disorders.

When administered as a prodrug, prodrugs of the compounds described herein can be administered in amounts sufficient to result in a dose of the compound of about 5 mg to about 2.5 g.

When used to treat or prevent the above diseases or disorders, compounds and/or pharmaceutical compositions thereof may be administered or applied singly, or in combination with other agents. Compounds and/or pharmaceutical compositions thereof may also be administered or applied singly, or in combination with other pharmaceutically active agents, including other compounds disclosed herein.

Methods of treatment and prophylaxis by administration to a patient of a therapeutically effective amount of a compound or pharmaceutical composition thereof are provided herein.

The present compounds and/or pharmaceutical compositions thereof, can be administered orally. Compounds and/or pharmaceutical compositions thereof may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) and can be used to administer compounds and/or pharmaceutical compositions thereof. Methods of administration include, but are not limited to, intradermal, intramuscular, intraperitoneal, intravenous, subcutaneous, intranasal, epidural, oral, sublingual, intranasal, buccal, intracerebral, intravaginal, transdermal, rectally, by inhalation, or topically, particularly to the ears, nose, eyes, or skin.

In certain embodiments, it may be desirable to introduce one or more compounds and/or pharmaceutical compositions thereof into the central nervous system by any suitable route, including intraventricular, intrathecal and epidural injection. Intraventricular injection may be facilitated by an intraventricular catheter, for example, attached to a reservoir, such as an Ommaya reservoir.

In some embodiments, compounds and/or pharmaceutical compositions thereof can be delivered via sustained release systems, such as oral sustained release systems. In some embodiments, a pump, including a micropump, may be used (see “Medical Applications of Controlled Release,” Langer and Wise (eds.), CRC Pres., Boca Raton, Fla. (1974); Sefton, 1987, CRC Crit Ref Biomed Eng. 14:201; Saudek et al., 1989, N. Engl. J. Med. 321:574).

In other embodiments, polymeric materials can be used (see “Medical Applications of Controlled Release,” Langer and Wise (eds.), CRC Pres., Boca Raton, Fla. (1974); “Controlled Drug Bioavailability,” Drug Product Design and Performance, Smolen and Ball (eds.), Wiley, New York (1984); Langer et al., 1983, J Macromol. Sci. Rev. Macromol Chem. 23:61; see also Levy et al., 1985, Science 228: 190; During et al., 1989, Ann. Neurol. 25:351; Howard et al., 1989, J. Neurosurg. 71:105). In still other embodiments, polymeric materials are used for oral sustained release delivery. Such polymers include, for example, sodium carboxymethylcellulose, hydroxypropylcellulose, hydroxypropylmethylcellulose and hydroxyethylcellulose. Other cellulose ethers have been described (Alderman, Int. J. Pharm. Tech. & Prod. Mfr. 1984, 5(3) 1-9). Factors affecting drug release are well known to the skilled artisan and have been described in the art (Bamba et al., Int. J. Pharm. 1979, 2, 307).

In other embodiments, enteric-coated preparations are used for oral sustained release administration. Coating materials include, for example, polymers with pH-dependent solubility (i.e., pH-controlled release), polymers with slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In still other embodiments, osmotic delivery systems are used for oral sustained release administration (Verma et al., Drug Dev. Ind. Pharm. 2000, 26:695-708). In some embodiments, OROS™ osmotic devices are used for oral sustained release delivery devices (Theeuwes et al., U.S. Pat. No. 3,845,770; Theeuwes et al., U.S. Pat. No. 3,916,899).

For administration by inhalation, compounds may be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas, may be used to deliver compounds disclosed herein directly to the lung.

Alternatively, a Dry Powder Inhaler (“DPI)” device may be used to administer compounds to the lung (See, e.g., Raleigh et al., Proc. Amer. Assoc. Cancer Research Annual Meeting, 1999, 40, 397). DPI devices typically use a mechanism such as a burst of gas to create a cloud of dry powder inside a container, which may then be inhaled by the patient. A popular variation is the multiple dose DPI (“MDDPI”) system, which allows for the delivery of more than one therapeutic dose. For example, capsules and cartridges of gelatin for use in an inhaler or insufflator may be formulated containing a powder mix of a compound and a suitable powder base such as lactose or starch for these systems.

Another type of device that may be used to deliver compounds to the lung is a liquid spray device supplied, for example, by Aradigm Corporation, Hayward, Calif. Liquid spray systems use extremely small nozzle holes to aerosolize liquid drug formulations that may then be directly inhaled into the lung.

In some embodiments, a nebulizer device is used to deliver a compound disclosed herein to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96). Examples of nebulizers include devices supplied by Batelle Pulmonary Therapeutics (Columbus, Ohio.) (See, Armer et al., U.S. Pat. No. 5,954,047; van der Linden et al., U.S. Pat. No. 5,950,619; van der Linden et al., U.S. Pat. No. 5,970,974).

In still other embodiments, an electrohydrodynamic (“EHD”) aerosol device is used to deliver a compound to the lung. EHD aerosol devices use electrical energy to aerosolize liquid drug solutions or suspensions (see e.g., Noakes et al., U.S. Pat. No. 4,765,539; Coffee, U.S. Pat. No. 4,962,885; Coffee, International Publication No. WO 94/12285; Coffee, International Publication No., WO 94/14543; Coffee, International Publication No., WO 95/26234, Coffee, International Publication No., WO 95/26235, Coffee, International Publication No., WO 95/32807). Electrochemical properties of a compound may be important parameters to optimize when delivering the compounds provided by the present disclosure to the lung with an EHD aerosol device; such optimization is routinely performed by one of skill in the art. EHD aerosol devices may more efficiently deliver compounds to the lung than existing pulmonary delivery technologies. Other methods of intra-pulmonary delivery of compounds are known to the skilled artisan and are within the scope of the present disclosure.

### Pharmaceutical Compositions

The present pharmaceutical compositions contain a therapeutically effective amount of one or more compounds, preferably in purified form, together with a suitable amount of a pharmaceutically acceptable vehicle, to provide the form for proper administration to a patient. When administered to a patient, the compounds and pharmaceutically acceptable vehicles are preferably sterile. In some embodiments, water is the vehicle when a compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions comprising a compound disclosed herein may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds into preparations which can be used pharmaceutically. Proper formulation is dependent upon the route of administration chosen.

The present compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use. In some embodiments, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 19th Edition, 1995). In other embodiments, pharmaceutical compositions are formulated for oral delivery.

Pharmaceutical compositions for oral delivery may be in the form of tablets, lozenges, aqueous or oily suspensions, granules, powders, emulsions, capsules, syrups, or elixirs, for example. Orally administered pharmaceutical compositions may contain one or more optional agents, for example, sweetening agents such as fructose, aspartame or saccharin, flavoring agents such as peppermint, oil of wintergreen, or cherry coloring agents and preserving agents, to provide a palatable pharmaceutical preparation. Moreover, where in tablet or pill form, the pharmaceutical compositions may be coated to delay disintegration and absorption in the gastrointestinal tract, thereby providing a sustained action over an extended period of time. Selectively permeable membranes surrounding an osmotically active driving compound are also suitable for orally administered compounds and pharmaceutical compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. In some embodiments, where in tablet or pill form, the pharmaceutical compositions are formulated to provide for immediate release.

In some embodiments, pharmaceutical compositions for oral delivery may be in form of tablets or capsules, for example, having 2.5 mg to 1000 mg of a compound described herein. In some embodiments, the pharmaceutical composition formulated for oral delivery comprises 2.5 mg, 5 mg, 10 mg, 25 mg, 50 mg, 100 mg, 250 mg, 500 mg, or 1000 mg of the compound.

In some embodiments, pharmaceutical compositions for oral delivery may be in form of tablets or capsules comprising two or more compound described herein. In some embodiments, the two or more compounds are present in the pharmaceutical composition in a ratio of 1:1. In some embodiments, the two or more compounds are present in the pharmaceutical composition in an uneven ration. In some embodiments, the pharmaceutical composition formulated for oral delivery comprises a combined total of 2.5 mg, 5 mg, 10 mg, 25 mg, 50 mg, 100 mg, 250 mg, 500 mg, or 1000 mg of the two or more compounds In some embodiments, the pharmaceutical composition formulated for oral delivery comprises 2.5 mg, 5 mg, 10 mg, 25 mg, 50 mg, 100 mg, 250 mg, 500 mg, or 1000 mg of each of the two or more compound.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM), etc. Additionally, flavoring agents, preservatives, coloring agents, bile salts, acylcarnitines and the like may be added.

Compounds may also be formulated in rectal or vaginal pharmaceutical compositions such as suppositories or retention enemas, e.g., containing conventional suppository bases such as cocoa butter or other glycerides.

In addition to the formulations described previously, compounds may also be formulated as a depot preparation. Such long acting formulations may be administered by implantation (for example, subcutaneously or intramuscularly), topically by a skin patch, or by intramuscular injection. Thus, for example, compounds may be formulated with suitable polymeric or hydrophobic materials (for example, as an emulsion in an acceptable oil) or ion exchange resins, or as sparingly soluble derivatives, for example, as a sparingly soluble salt.

When a compound is acidic, it may be included in any of the above-described formulations as the free acid, a pharmaceutically acceptable salt, a solvate or hydrate. Pharmaceutically acceptable salts substantially retain the activity of the free acid, may be prepared by reaction with bases and tend to be more soluble in aqueous and other protic solvents than the corresponding free acid form.

Liquid drug formulations suitable for use with nebulizers and liquid spray devices and EHD aerosol devices will typically include a compound with a pharmaceutically acceptable carrier. The pharmaceutically acceptable carrier may be a liquid such as alcohol, water, polyethylene glycol or perfluorocarbon. Optionally, another material may be added to alter the aerosol properties of the solution or suspension of compounds disclosed herein. Preferably, this material is liquid such as an alcohol, glycol, polyglycol or fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611).

### Combination Therapy

In certain embodiments, the compounds disclosed herein can be used in combination therapy with at least one other therapeutic agent. The compound and the therapeutic agent can act additively or synergistically. In some embodiments, a pharmaceutical composition comprising a compound disclosed herein is administered concurrently with the administration of another therapeutic agent, which can be part of the same pharmaceutical composition or a different pharmaceutical composition. In other embodiments, a pharmaceutical composition comprising a compound disclosed herein is administered prior or subsequent to administration of another therapeutic agent.

### Phenotype-Based High-Throughput Chemical Screens

Identifying brain-penetrant neuroactive drugs using high-throughput screens in cellular systems is challenging and requires multiple optimization steps, which do not always result in more effective drugs. At the same time, applying high-throughput screening methodologies to rodent disease models is time consuming, costly, and in many cases, technologically impossible. In contrast, phenotype-based small molecule drug screens using zebrafish larvae provide a platform for large-scale in vivo drug discovery that merges the advantages of high-throughput cellular systems with the high-content of rodent disease models. Additionally, although there are marked differences between human and zebrafish behaviors, the neural systems of these two vertebrates exhibit a high degree of conservation both genetically and in cell signalling pathways.

Larval and adult zebrafish exhibit a rich repertoire of screenable behavioral phenotypes, including responses to anxiolytics, anxiogenics, antipsychotics, and pro- and anti-convulsants. The ability to analyze the swimming behaviors of zebrafish larvae in response to a given small molecule stimulus led to the development of several platforms for behavior-based drug screens. For example, a medium throughput screen was used to find compounds with anti-seizure properties among a collection of FDA-approved and toxicology tested compounds. Moreover, large-scale chemical screens for small molecule regulators of zebrafish behaviors like sleep, the photomotor response (PMR), and responses to antipsychotics has allowed for the rapid identification of novel neuroactive compounds and led to the successful predictions of their underlying target pathways.

Some embodiments provide materials and methods for performing phenotype-based chemical screens. Embodiments disclosed herein pertain to the use of a battery of high-throughput zebrafish behavioral assays to identify novel small molecule regulators of neurotransmission. In particular, embodiments of the present disclosure include performing high-throughput small molecule suppressor screens to identify novel small molecule suppressors of behavioral phenotypes generated from exposure to compounds that cause neuronal hyperexcitability, including but not limited to, inhibitors of the potassium chloride cotransporter, KCC2.

Generally, hyperpolarizing synaptic inhibition, such as that which occurs via the gamma-aminobutyric acid receptor type A (GABA_{A}R) and the glycine receptor (GlyR), is modulated by the intracellular concentration of chloride ions (Cl—) in postsynaptic neurons. The low Cl— concentration required for this inhibition is predominately established and maintained in the mature brain by potassium chloride cotransporters such as KCC2, which is encoded by the SLC12A5 gene. KCC2 extrudes Cl— primarily from CNS neurons, stabilizing inhibitory neurotransmission under normal conditions. Consistent with its importance to synaptic inhibition, KCC2 deficiencies have been linked to epilepsy in rodents and human patients. KCC2 variants have been identified in subjects with fever-induced and generalized epilepsies, as well as severe infantile-onset epilepsy. Moreover, pharmacological inhibition of KCC2 has been shown to induce epileptiform discharges in hippocampal slices and in the mouse hippocampus. KCC2 loss-of-function has also been reported in other neurological disorders that involve impaired GABA_{A} inhibition and synaptic hyperexcitability, including but not limited to, neuropathic pain, spinal cord injury spasticity, morphine-induced hyperalgesia, schizophrenia, and autism spectrum disorders. Therefore, it is not surprising that enhancing KCC2 activity can be a potentially effective strategy for restoring GABA_{A} neurotransmission as a treatment for these pathologies. However, despite this basic understanding and recent efforts do identify KCC2 enhancers in an effort to improve outcomes in these disease settings, there are no viable therapeutic options currently in development.

In some embodiments of the present disclosure, the effects of the selective inhibitors of KCC2, VU0463271 (N-Cyclopropyl-N-(4-methyl-2-thiazolyl)-2-[(6-phenyl-3-pyridazinyl)thio]acetamide, CAS No. 1391737-01-1) and VU0240551 (N-(4-Methyl-2-thiazolyl)-2-[(6-phenyl-3-pyridazinyl)thio]acetamide, CAS No. 893990-34-6), on zebrafish swimming behaviors at 7 days post-fertilization (dpf) were investigated. Generally, pharmacological inhibition of KCC2 using VU0463271 or VU0240551 produced a unique behavioral profile that was found to be distinct from the behavioral profile induced by pentylentetrazol or PTZ. Primary suppressor screens were performed using small molecule libraries containing compounds in an effort to identify suppressors of the inhibitor-induced behavioral phenotype (“rescue” wildtype or untreated behavior) produced by exposing zebrafish larvae to VU0463271 or VU0240551. As disclosed herein, three hit compounds and various derivatives were identified: TT0023831, TT0042607 and TT0011235, all of which rescued the effect of KCC2 inhibition in the behavioral profiles disclosed herein. These three hit compounds, each having a chemically distinct structure and exhibits distinct behavior phenotypes.

In some embodiments, methods provided by the present disclosure include subjecting organisms to a battery of behavioral assays to generate a behavioral profile. The behavioral assay can be carried out using various experimental parameters, such as exposing the organisms to different stimuli and quantifying the corresponding behavioral response. Any observable and quantifiable behavior can be used as the basis for a behavioral assay. For example, behavioral assays can be based on: a photomotor response in which the applied stimulus is a pulse of bright light; sleep patterns in which the applied stimulus involves changing the photoperiod; habituation in which the applied stimulus involves modulation of high-frequency stimulus trains; associative learning and extinction in which the applied stimulus involves the pairing of conditioned and unconditioned stimuli; application of a painful stimulus; the acoustic startle response in which the applied stimulus is an acoustic stimulus the visual startle response in which the applied stimulus is a visual stimulus; and combinations of any of the foregoing, among others.

According to the present disclosure, behavioral assays can be based on various combinations of stimuli applied in succession for specific durations and intensities. For example, a behavioral assay can include the application of acoustic stimuli, electric stimuli, visual stimuli, and the like for specific amounts of time and at specific intensities. Stimuli can be applied sequentially in any order or combination, with or without the addition of refractory periods, as one of skill in the art would readily appreciate based on the present disclosure. Various other stimuli can also be applied, and are not limited to those mentioned above, depending on the organism used and the experimental conditions, as one of ordinary skill would readily appreciate based on the present disclosure. In some cases, a stimulus can be applied for 0.1 second, 0.2 seconds, 0.3 seconds, 0.4 seconds, 0.5 seconds, 0.6 seconds, 0.7 seconds, 0.8 seconds, 0.9 seconds, 1 second, 2 seconds, 3 seconds, 4 seconds, 5 seconds, 6 seconds, 7 seconds, 8 seconds, 9 seconds, 10 seconds, 11, seconds, 12 seconds, 13 seconds, 14 seconds, 15 seconds, 16 seconds, 17 seconds, 18 seconds 19 seconds, 20 seconds, 21 seconds, 22 seconds, 23 seconds, 24 seconds, 25 seconds, 26 seconds, 27 seconds, 28 seconds, 29 seconds, 30 seconds, and/or any number of seconds up to and including 5 minutes. Stimuli can also be applied multiple times in succession for seconds or sub-second intervals.

Light-based stimuli can be applied by any means and can include light delivered at any wavelength that is capable of causing a response in an organism, including but not limited to red light (600 nm), blue light (420 nm), violet light (405 nm), and green light (525 nm). Light-based stimuli can also be applied multiple times in succession for seconds or sub-second intervals, with or without refractory periods. Electrical stimuli can be applied by any means and can include electricity delivered at 1 volt (V), 2V, 3V, 4V, 5V, 6V, 7V, 8V, 9V, 10V, 11V, 12V, 13V, 14V, 15V, 16V, 17V, 18V, 19V, 20V, 21V, 22V, 23V, 24V, 25V, and/or any amount of volts up to and including 50V. Electrical stimuli can also be applied multiple times in succession for seconds or sub-second intervals, with or without refractory periods. Acoustic stimuli can be applied by any means and can include electricity delivered at 1 volt (V), 2V, 3V, 4V, 5V, 6V, 7V, 8V, 9V, 10V, 11V, 12V, 13V, 14V, 15V, 16V, 17V, 18V, 19V, 20V, 21V, 22V, 23V, 24V, 25V, and/or any amount of volts up to and including 50V. Electrical stimuli can also be applied multiple times in succession for seconds or sub-second intervals, with or without refractory periods.

Any number of behavioral assays can be used to generate a behavioral profile. A behavioral profile can include a single behavioral assay, 2 behavioral assays, 3 behavioral assays, 4 behavioral assays, 5 behavioral assays, 6 behavioral assays, 7 behavioral assays, 8 behavioral assays, 9 behavioral assays, 10 behavioral assays, and as many as 20 behavioral assays. The number and type of behavioral assays that make up a behavioral profile can readily be determined by one of skill in the art based on the present disclosure, and can be depend on such factors as, for example, the organism being used and the experimental parameters being investigated.

In an effort to determine how pharmacological inhibition of KCC2 affects zebrafish behaviors, 7 days post-fertilization (dpf) zebrafish larvae can be treated with two analogs of the selective KCC2 inhibitor, VU0463271 and VU0240551, and their behavioral responses in a battery of behavioral assays can be assessed. These behavioral assays can include a series of visual and acoustic stimuli presented to larval zebrafish in various contexts and combinations to elicit robust and reproducible patterns of activity. For example, behavioral assays can include two acoustic-stimulus-response (ASR1 and ASR2) assays, six visual-stimulus-response (VSR1-VSR6) assays, and three assays that combine acoustic and visual stimulus responses (AVSR1-AVSR3). Together, such a battery of behavioral assays use six stimuli, including red light (600 nm), blue light (420 nm), violet light (405 nm), green light (525 nm), low-intensity sound (60 dB) and high-intensity sound (70 dB). Behavioral responses of the larval zebrafish can be measured by a motion index (MI) that quantifies the motor activity of at least about 8 zebrafish larvae in each well of a 96-well plate.

It is often difficult to predict what types of a chemical compounds a particular behavioral assay will identify. A behavioral assay using a particular stimulus may identify compounds with biological functions that do not appear to correlate with the compounds' identified biological activity in a vertebrate animal (e g., a human). With sufficiently rich behavioral phenotyping, however, the present disclosure provides ways to identify mechanistic relationships between molecules within a large collection simply on the basis of their phenotypic similarity. This approach, based solely on the behavioral effects produced by various chemicals, can provide an unbiased, structure- and target-independent platform for identifying novel therapeutic compounds.

Embodiments of the present disclosure can be applicable to the discovery of therapeutic agents for the treatment of any disease or disorder. Embodiments of the present disclosure are particularly well suited to the discovery of novel therapeutic agents for the treatment of CNS disorders and diseases, due in part to the polygenic nature of these disorders as well as the dearth of known targets underlying most CNS disorders and diseases. CNS disorders can be drug induced, can be attributed to genetic predisposition, infection or trauma, or can be of unknown etiology. Materials and methods of the present disclosure can be used to treat CNS disorders, including but not limited to neuropsychiatric disorders, neurological diseases and mental illnesses, neurodegenerative diseases, behavioral disorders, cognitive disorders, and cognitive affective disorders. There are several CNS disorders whose clinical manifestations have been attributed to CNS dysfunction (e.g., disorders resulting from inappropriate levels of neurotransmitter release, inappropriate properties of neurotransmitter receptors, and or inappropriate interaction between neurotransmitters and neurotransmitter receptors). Several CNS disorders can be attributed to a cholinergic deficiency, a dopaminergic deficiency, an adrenergic deficiency and/or a serotonergic deficiency. CNS disorders of relatively common occurrence include presenile dementia (early onset Alzheimer's disease), senile dementia (dementia of the Alzheimer's type), Parkinsonism including Parkinson's disease, Huntington's chorea, tardive dyskinesia, hyperkinesia, mania, attention deficit disorder, anxiety, dyslexia, schizophrenia, psychosis, bipolar disorder, depression, and Tourette's syndrome, as well as epilepsy, sleep disorders, ataxia, hearing and vision disorders, autism spectrum disorders and pain. Embodiments of the present disclosure can be used to discover therapeutic agents for the treatment of any of the aforementioned CNS disorders, as one of ordinary skill would readily appreciate based on the present disclosure.

Embodiments of the present disclosure may include subjecting organisms to a battery of behavioral assays after exposing the organisms to an experimental perturbation. The experimental perturbation may exert an effect on the organisms which may be reflected in the behavioral assay and/or the behavioral profile. For example, behavioral assays can be conducted on organisms exposed to a chemical compound from a chemical library. In some cases, the chemical compounds used as experimental perturbations can have known or unknown biological functions. In some cases, the chemical compounds can have known biological functions, including but not limited to, psychoactive properties.

In addition to perturbations caused by chemical compounds, embodiments of the present disclosure can also be used to create behavioral profiles in the context of genetic or environmental perturbations. For example, materials and methods of the present disclosure can be used to create databases containing behavioral profiles of organisms with known or unknown genetic defects or mutations, which can be compared to the behavioral profiles of organisms with known genetic defects or mutations or wild type organisms, in an effort to link phenotypic similarities and identify a novel gene or genes contributing to a given phenotype. In some aspects, the underlying phenotype can mimic a human disease phenotype. In other aspects, the genetic mutation can be in a gene known to contribute to the disease phenotype in a human. Other experimental perturbations can include genetic mutations causing various quantifiable phenotypes, including transgenics, hypermorphs, hypomorphs, loss-of-function, and gain-of-function mutants. Mutations can occur at coding and non-coding loci. Behavioral assays can also be conducted to determine if chemical compounds are able to suppress a given phenotype induced by a genetic or environmental perturbation or another chemical compound (suppressor screen). Behavioral assays can also be created around various environmental perturbations (e.g., oxygen levels, relative pressures, degrees of heat/cold, radiation exposure, etc.).

Embodiments of the present disclosure may include a high-throughput screening (HTS) platform for conducting the behavioral assays and generating quantifiable summaries of the behavioral profiles. HTS methods generally involve the use of robotics, data processing and control software, liquid handling devices, and sensitive detectors. The HTS methods of the present disclosure allow researchers to quickly conduct millions of biochemical, genetic or pharmacological assays and generate high-content data using whole organisms. The HTS platform of the present disclosure generally includes both hardware and software. The software is designed to control the hardware and analyze images captured of the behavior of the organisms during the behavioral assays. In some cases, the software synchronizes image acquisition with the presentation of one or more stimuli. The software can save, store, and organize the raw image data, extract behavioral data from the images and analyze the data for specific characteristics (e.g., common phenotypic characteristics). The software can also take these data and generate a quantitative summary or quantitative summaries of the behavioral profiles, which can be compared to another quantitative summary or other quantitative summaries, analyzed for similarities and differences, and stored in a database. A behavioral profile can be generated for each experimental perturbation (e.g., chemical or genetic). In some cases, the behavioral profiles (also known as behavioral barcodes) can be used to identify chemical compounds with a desired set of activities or biological effects, and also to predict biological mechanisms of action.

Integrated systems for use with the chemical screening platform of the present disclosure can comprise one or more automated devices for loading microtiter plates containing various organisms and/or detectable label(s), and/or test agent(s), and/or automated devices for reading the results of the assay. Integrated systems can also include additional robotics for sample processing, reagent synthesis, microtiter plate storage and/or incubation and/or handling, computer systems for controlling the devices and recording and/or analyzing assay data and the like. Other electronic hardware and/or software may be implemented to enhance image acquisition, data storage and/or synchronization, as one skilled in the art would appreciate from the present disclosure.

Once a phenotype-based chemical screen has been conducted, a quantitative summary or quantitative summaries corresponding to the behavioral profile(s) generated can be analyzed for common phenotypic characteristics. These data can be clustered to determine structural similarities among the chemical compounds identified as having, for example, similar behavioral profiles. In some cases, the data can be queried using a blast-type approach (e.g., phenoBlast) in that a reference compound with known biological functions can be compared to chemical compounds with unknown biological functions to identify those compounds with similar behavioral profiles.

Zebrafish embryos or zebrafish larva are well suited for conducting phenotype-based chemical screens. During the embryonic and larval stages of life, the zebrafish is only about 1-2 mm long, and can live for days in a single well of a standard 96-well or a standard 384-well plate, surviving on nutrients stored in its yolk sac. Chemical compounds can also easily diffuse and penetrate the embryonic and larval zebrafish body. The genome and body plan of the zebrafish are similar to those of other vertebrates, and its optical transparency and external development enable real time observation of its internal organs and physiological systems. The optical clarity of the zebrafish embryo becomes even more useful when combined with fluorescent markers that highlight the locations or activities of specific populations of cells. For example, dozens of transgenic zebrafish lines have been created which express fluorescent proteins in many different organs, tissues and cells in vivo. Transgenic zebrafish lines greatly facilitate detection of behavioral changes caused by small molecules. Numerous zebrafish disease models ranging from congenital heart defects to cancers have been developed, and the zebrafish is genetically and pharmacologically similar to humans. Further, because screening can be performed in the whole organism, perturbation of potential therapeutic targets by small molecules or mutations reveals the effects of such perturbations on the integrated physiology of the entire organism.

In some embodiments, zebrafish larva between about 2 days and 10 days post-fertilization (e.g., obtained by mating Ekkwill zebrafish) can be transferred to wells of clear, flat-bottom and square 96-well plates filled with embryo liquid medium (e.g., 300 μl of E3 medium) prior to entry of the plates into an automated high-throughput screening platform. In some cases, the stimuli applied during a behavioral assay as well as the digital recordings of the assay can be applied to the entire 96-well plate simultaneously. In some cases, each behavioral assay, including video recording and data processing, can occur in about 40s per plate (per 30s assay); a battery of 10 behavioral assays using 96-well plates can be screened in less than 10 minutes.

### EXAMPLES

The following examples further define the disclosure and describe in detail preparation of compounds, pharmaceutical compositions thereof and assays for using compounds and pharmaceutical compositions. It will be apparent to those skilled in the art that many modifications, both to materials and methods, may be practiced without departing from the scope of the disclosed subject matter.

### Example 1

### Treatment of Zebrafish Larvae with Pharmacological Inhibitors of KCC2 Produces Unique Behavioral Phenotypes

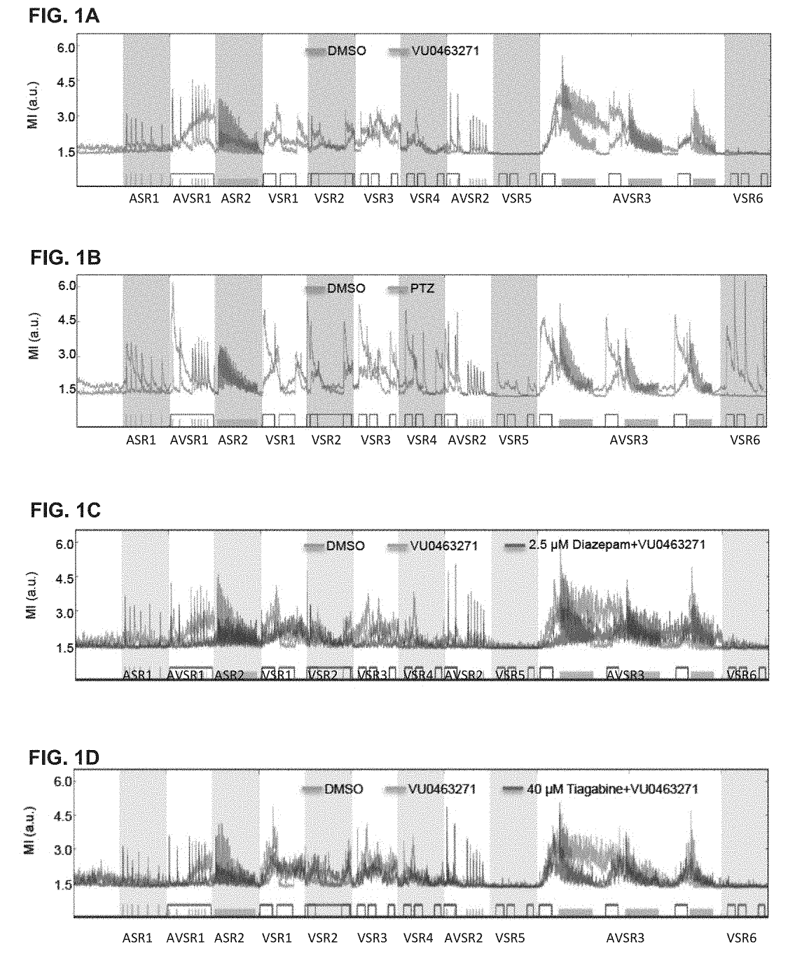

Referring to FIGS. 1A-1B, zebrafish behavioral profiles can include a battery of individual behavioral assays labeled ASR1, AVSR1, ASR2, VSR1, VSR2, VSR3, VSR4, AVSR2, VSR5, AVSR3, and VSR6, as described herein. These behavioral assays can be combined to form a Motion Index (MI), which is a quantification of the motor activity of at least about 8 zebrafish larvae in each well of a 96-well plate. As shown in the representative images of FIGS. 1A-1B, zebrafish larvae were treated with DMSO (control), VU0463271 (FIG. 1A), or pentylentetrazol or PTZ (FIG. 1B).

Treatment with increasing doses of VU0463271 induced a unique and dose-dependent profile of behavioral responses, detectable at 2.5 μM and 5 μM. At 10 μM, VU0463271 had a sedative effect, leading to a very low MI along the assay battery. Treatment with 5-40 μM VU0240551 induced a similar but not identical profile of behavioral responses to that induced by VU0463271. To generate a high-resolution behavioral profile of KCC2 inhibition in the most effective but non-sedative concentrations, zebrafish larvae in 48 wells of a 96-well plate were treated with 2.5 μM VU0463271, and zebrafish larvae in the other 48 wells were treated with DMSO (FIG. 1A). This relatively low concentration of VU0463271 elicited a reproducible behavioral profile with altered sensitivity of the fish larvae to specific combinations of stimuli. For example, treatment increased behavioral responses in the AVSR1 and AVSR3 assays and reduced the MI in the ASR2 and VSR assays generally. The increased MI in the AVSR1 and AVSR3 assays due to VU0463271 treatment reflects a seizure-like hyperexcitability in the larval fish. For example, the failure of the larval fish to habituate to the repetitive acoustic stimulation in the AVSR3 assays (FIG. 1A) is a similar behavioral phenotype as that of the repeatedly induced-seizures in the kindling model of epilepsy, and is consistent with the recent finding that VU0463271 can increase the duration of seizure-like events (SLEs) in mouse brain slices.

Additionally, seizures have been induced in 7 dpf zebrafish larvae using pentylentetrazol (PTZ) treatment, and motor and electroencephalographic (EEG) responses were consistent with seizure phenotypes. As depicted in FIG. 1B, 5 mM PTZ induced hyperactivity and a dramatic increase in the MI in response to multiple light and acoustic stimuli. The shape of the behavioral profiles of fish treated with KCC2 inhibitors is distinct from behavioral profiles of fish treated with PTZ, although fish treated with either exhibited elevated and sustained responses to specific stimuli, consistent with neuronal hyperexcitability.

Therefore, pharmacological inhibition of KCC2 in zebrafish produces a distinctive pattern of behavioral perturbations that can be useful for discovering and characterizing novel modulators of hyperexcitability.

### Example 2

### Phenotypic Effects of Treatment with Pharmacological Inhibitors of KCC2 can be Rescued in Zebrafish Larvae

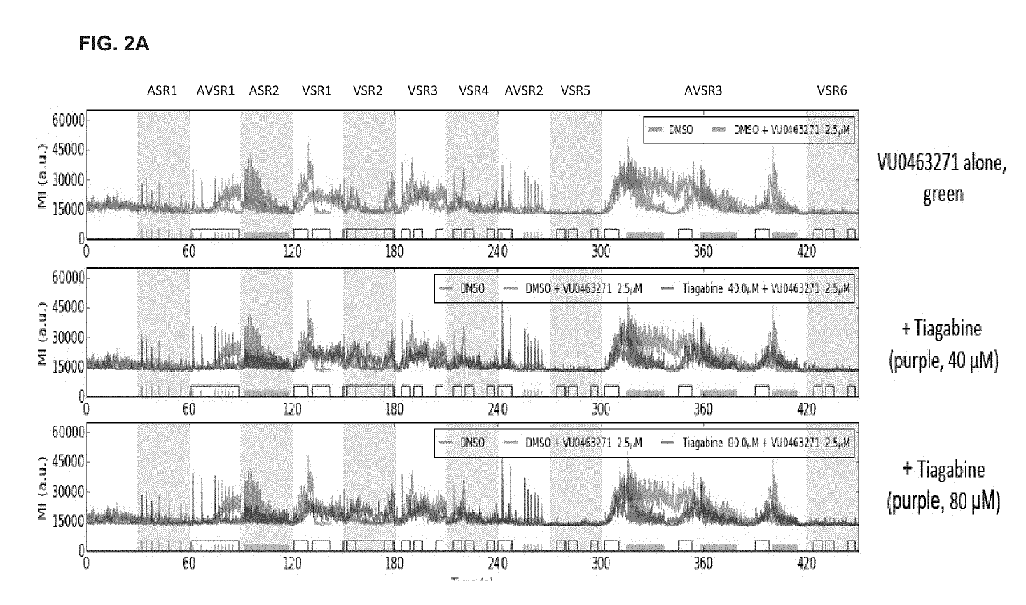

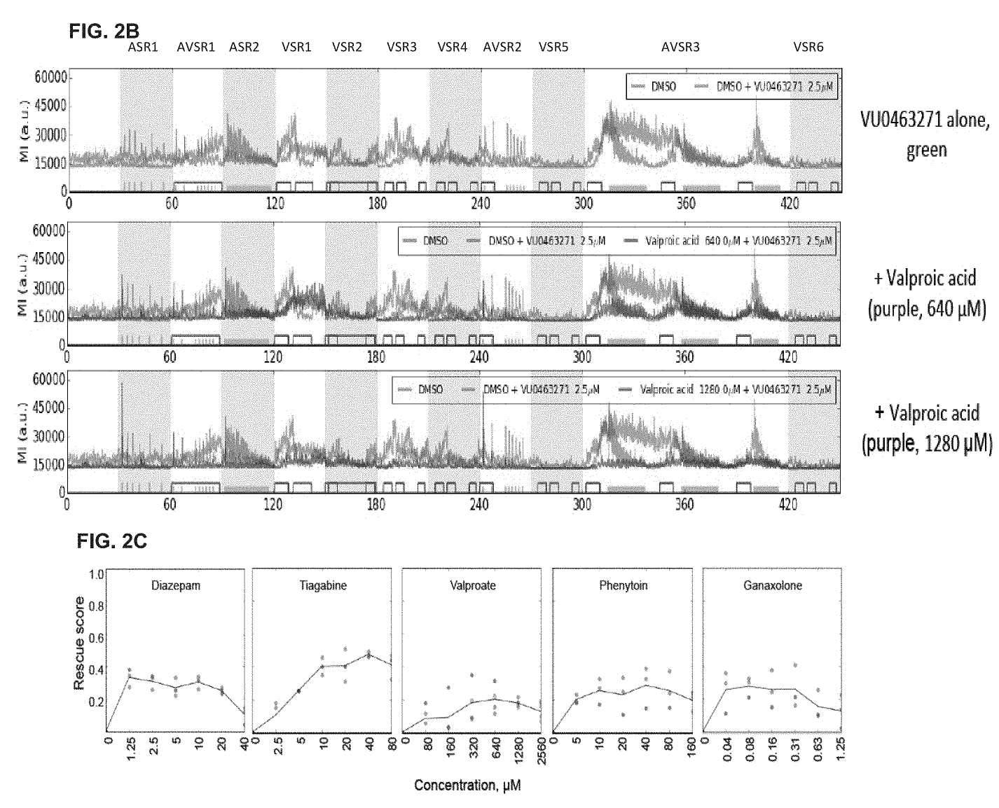

Referring to FIGS. 1A-1D and FIGS. 2A-2C, zebrafish behavioral profiles can include a battery of individual behavioral assays labeled ASR1, AVSR1, ASR2, VSR1, VSR2, VSR3, VSR4, AVSR2, VSR5, AVSR3, and VSR6, as described herein. Behavioral assays can be combined to form a Motion Index (MI), which is a quantification of the motor activity of at least about 8 zebrafish larvae in each well of a 96-well plate. As depicted in these representative images, zebrafish larvae were treated with DMSO (control), VU0463271±Diazepam (FIG. 1C), VU0463271±Tiagabine (FIG. 1D and FIG. 2A), or VU0463271±Valproic acid.

Differences between behavioral profiles of larval zebrafish treated with KCC2 inhibitors and larval zebrafish treated with PTZ were investigated by evaluating the ability of traditional anti-seizure drugs to reverse their effects. Five drugs in at least six concentrations (by allocating 12 wells per concentration) were tested. These drugs included a benzodiazepine anxiolytic (diazepam), two AEDs that increase GABA levels in the synapse (tiagabine and valproate), a sodium channel blocker (phenytoin) and a synthetic neurosteroid GABAA positive allosteric modulator (PAM, ganaxolone). It was found that pre-treatment with non-sedative concentrations of either diazepam (2.5 μM), tiagabine (40 μM), valproate (640 μM) or ganaxolone (0.31 μM) almost completely prevented the formation of the PTZ-induced behavior, whereas phenytoin had almost no effect (160 μM) (data not shown).

To quantify the ability of the same drugs to reverse (or “rescue”) the phenotype caused by either 2.5 μM VU0463271 or 5 μM VU0240551, a rescue score between 0 (no rescue) and 1 (maximal theoretical rescue) was calculated (FIG. 2C). A rescue score incorporates and quantifies the degree of phenotypic reversal and the level of sedation in each concentration tested (see Materials and Methods below). It was found that tiagabine was the most effective at rescuing the behavioral profile induced by KCC2 inhibition back to the normal response (FIG. 1D, and FIGS. 2A and 2C). As shown in FIG. 2C, the other four drugs only partially rescued the phenotype and, at higher concentrations, these drugs caused sedation, which contributed to lower rescue scores (FIG. 1C for diazepam; FIG. 2B for valproic acid). FIG. 2A is a representative image demonstrating the ability of tiagabine acid to suppress the VU0463271-induced phenotype; and FIG. 2B is a representative image demonstrating the ability of valproic acid to suppress the VU0463271-induced phenotype.

These data demonstrate the sensitivity and selectivity of the behavioral assays and behavioral profiles of the present disclosure to identify modulators of a hyperexcitability phenotype. Additionally, the lower efficacies of the four anti-seizure drugs to rescue the KCC2 inhibitor-induced behavioral profiles are consistent with the inability of these established AEDs to provide relief only for certain sub-populations of epilepsy patients.

### Example 3

### Small Molecule Suppressor Screens for Modulators of Behavioral Phenotypes Produced by KCC2 Inhibition

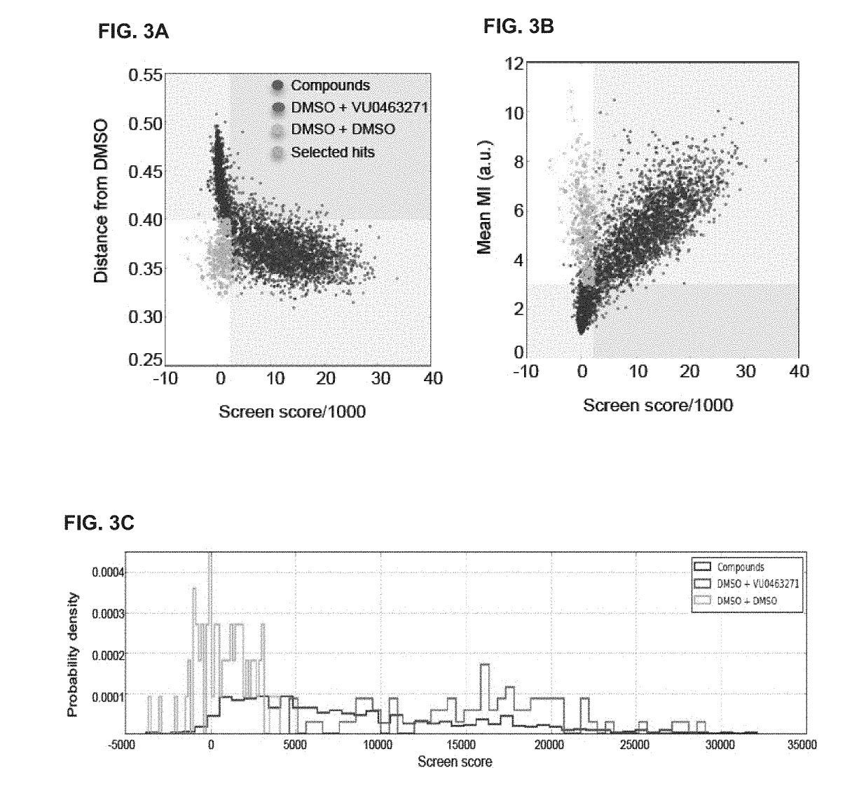

Referring to FIGS. 3A-3C, representative scatter plots (FIGS. 3A-3B) and histograms (FIG. 3C) demonstrating the ability of various compounds to suppress or rescue the VU0463271-induced phenotype are depicted. Based on the robust behavioral phenotypes of the selective KCC2 inhibitors and the partial suppression of these behaviors by known anti-seizure drugs and anxiolytics, the behavioral profiles observed with these inhibitors were used to identify novel suppressors from a chemical library of unannotated compounds. A diverse collection of 37,000 uncharacterized small molecules were used in a suppressor screen against either 2.5 μM VU0463271 (27,000 compounds) or 5 μM VU0240551 (10,000 compounds), along with 3,700 control wells treated with DMSO alone and 3,700 control wells treated with the KCC2 inhibitor. Each well of a 96-well plate containing at least about 8 zebrafish larvae was treated with a library compound or DMSO followed by treatment with the KCC2 inhibitor. Behavioral profiles pre- and post-treatment with the KCC2 inhibitor were evaluated using the behavioral assay battery described herein, and a detailed quantitative readout of about 15,000 time points was generated for each well. These behavioral profiles were used to calculate a screening score for each library compound, which incorporates and quantifies the rescue score and sedation index (see Materials and Methods).

In some cases, small molecules that alter the KCC2 inhibition phenotype may reverse the behavioral phenotype back to the normal, but could also elicit a new undesired phenotype, which is different from a normal behavior. To discriminate between these possibilities, vector algebra was used to compare each behavioral profile to the behavioral profile of DMSO-treated larvae and defined a value for the phenotypic distance from DMSO. The screen score of each compound was then plotted against its averaged phenotypic distance from DMSO (FIG. 3A) and MI (FIG. 3B) to filter out compounds that elicit a profile that is very different from the control one and compounds that reduce overall activity, respectively.

A total of 320 compounds with scores <0, phenotypic distances from DMSO<0.4 and mean MI>3 were identified as potential hits for retesting. Using these criteria, 240 compounds identified from the VU0463271 suppressor screen and 80 compounds identified from the VU0240551 suppressor screen were selected. These selected compounds were retested for their ability to rescue the effect of the KCC2 inhibitor, and screen scores were recalculated for each compound. A probability density analysis of the resulting scores showed that the behavioral profiles of larval zebrafish treated with a KCC2 inhibitor are well separated from those of fish treated with DMSO alone (FIG. 3C), confirming the robustness and reproducibility of the phenotype. The distribution of the selected hits was wider, overlapping with both DMSO and the KCC2 inhibitor. Among these, 30 compounds were identified with a distribution that overlaps with the DMSO control, but not with the KCC2 inhibitor. These compounds were selected for reordering and further testing.

Overall, these data demonstrate the sensitivity and selectivity of the behavioral assays and behavioral profiles of the present disclosure useful for discovering and characterizing novel small molecule modulators of hyperexcitability.

### Example 4

### Identification of Compounds that Rescue Behavioral Phenotypes Produced by KCC2 Inhibition

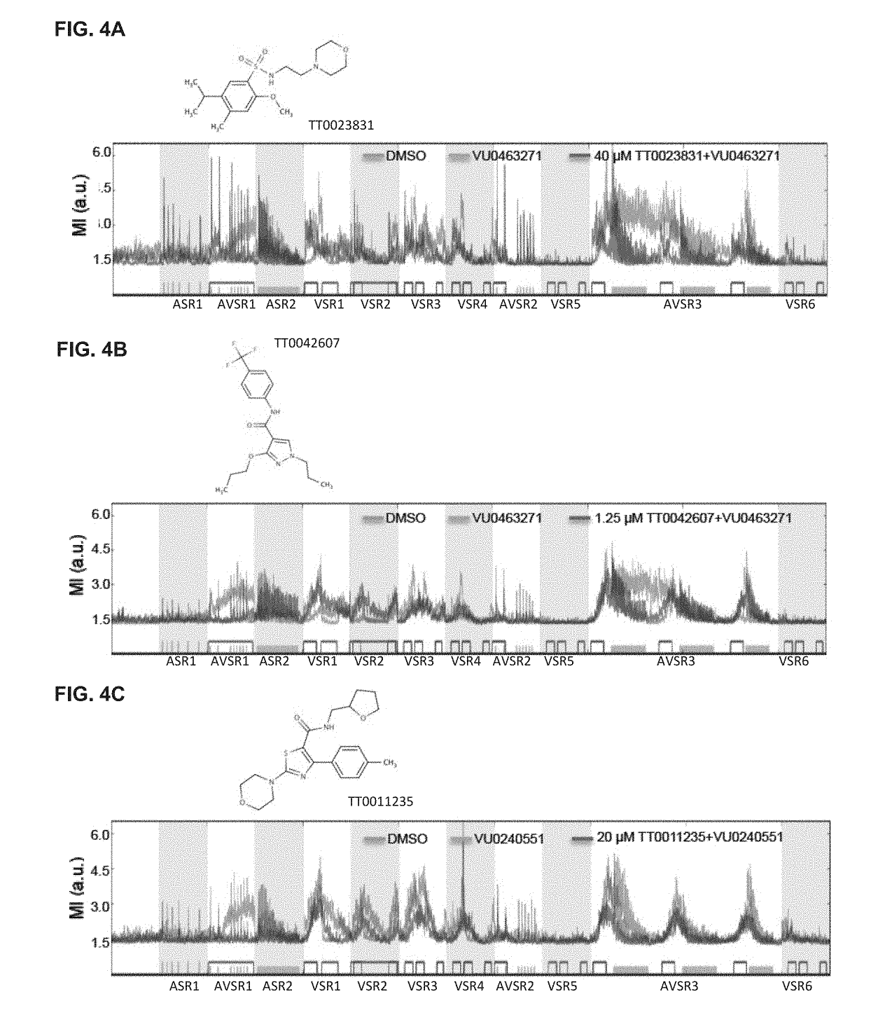

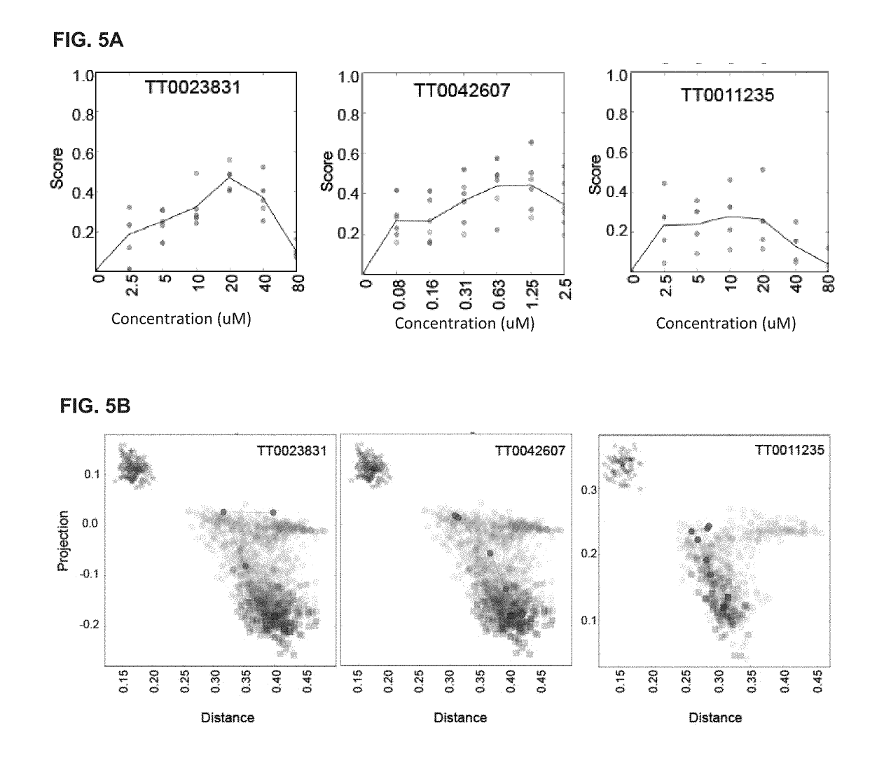

Referring to FIGS. 4A-4C, representative behavioral profiles (average MI) of the top three small molecule modifiers identified in primary screens, along with their corresponding chemical structures are presented. TT0023831 (FIG. 4A) and TT0042607 (FIG. 4B) were identified in a VU0463271 suppressor screen, while TT0011235 was identified in a VU0240551 suppressor screen (FIG. 4C). Additionally, referring to FIGS. 5A-5B, representative line graphs (FIG. 5A) and scatter plots (FIG. 5B) demonstrating the ability of three hit compounds to suppress VU0463271 or VU0240551-induced phenotypes are shown.

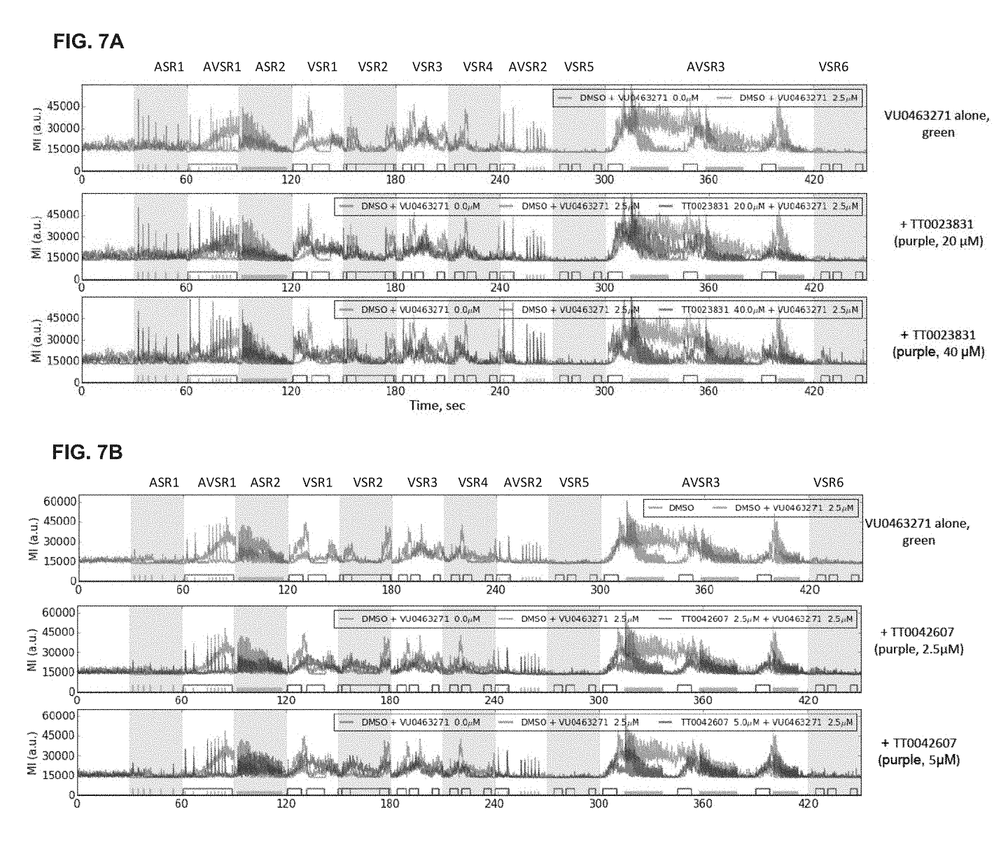

The reproducibility of the effects and the active concentration ranges of the 30 hits selected, as described above, were determined. Their rescue efficacies were retested in at least six concentrations and their rescue scores were also recalculated. The top three hit compounds represented three structural classes: TT0023831 and TT0042607 that were identified in the VU0463271 screen and TT0011235 that was identified in the VU0240551 screen (FIGS. 4A-4C). For each hit compound, the traces of the averaged MI signatures were analyzed (FIGS. 4A-4C), the rescue scores for each concentration were calculated (FIG. 5A), and a 2D scatter plot demonstrating the projection of the behavioral profile from a VU0463271 or VU0240551-induced profiles toward the collection of the DMSO control profiles was established (FIG. 5B). Additionally, FIGS. 7A-7B are images of representative behavioral profiles (average MI) of TT0023831 (FIG. 7A) and TT0042607 (FIG. 7B), and their ability to suppress the VU0463271-induced phenotype at the concentrations indicated.

It was found that TT0023831 (tested at 2.5-80 μM) displayed the best phenotypic reversal at 20 μM and 40 μM, evidenced by high rescue scores (FIG. 5A) and a dose-dependent projection toward the DMSO control profiles (FIG. 5B). Treatment with 80 μM TT0023831 reduced the overall motor activity of the larvae, which in turn reduced the rescue score. Treatment with TT0042607 (tested at 0.08-2.5 μM) rescued the VU0463271-induced behaviors already at sub-micromolar concentrations and had no effect on the overall MI in these or higher concentrations (FIGS. 5A-5B). Treatment with TT0011235 (tested at 2.5-80 μM) rescued the VU0240551-induced behaviors, with the most effective phenotypic reversal at 20 μM and an inhibitory effect on overall MI at higher concentrations (FIGS. 5A-5B).

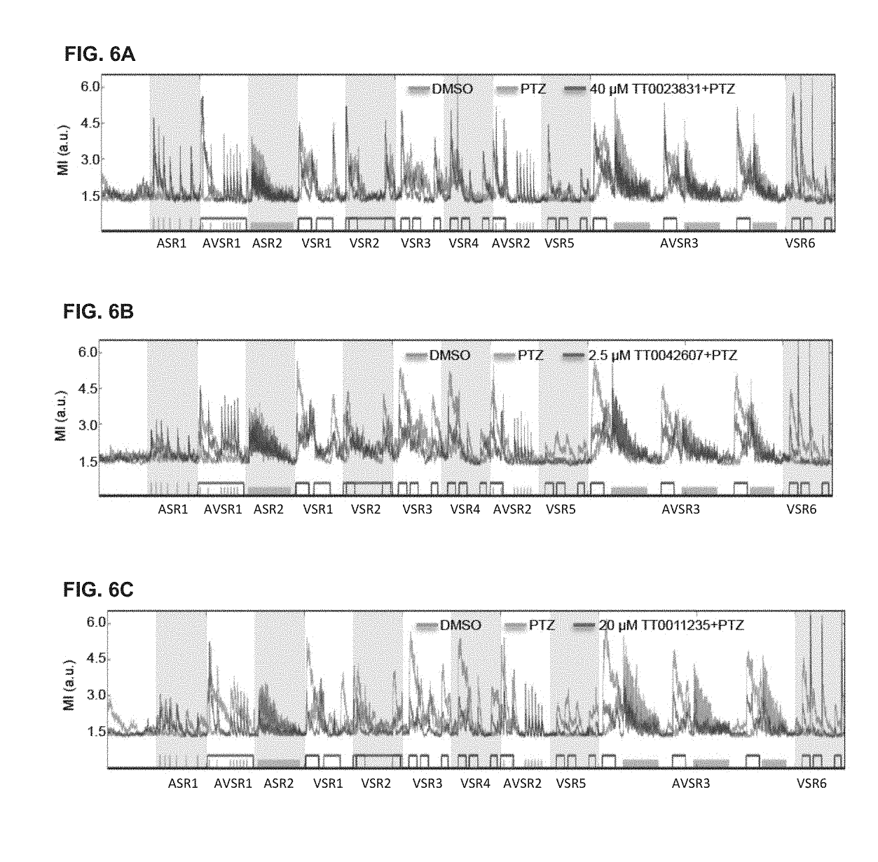

To investigate further the ability of these three small molecule hits to act as anti-seizure compounds, their ability to suppress the PTZ-induced behavior in zebrafish larvae was evaluated. As was previously performed with the reference anti-seizure drugs, zebrafish larvae were pre-treated with 6 concentrations of the three hit compounds, followed by 5 mM PTZ treatment. It was found that the three hit compounds yielded different effects. Both TT0042607 and TT0011235 attenuated PTZ-induced hyperactivity in response to the different stimuli, whereas TT0023831 did not (FIGS. 6A-6C), suggesting distinct mechanisms of action for these three chemical structures.

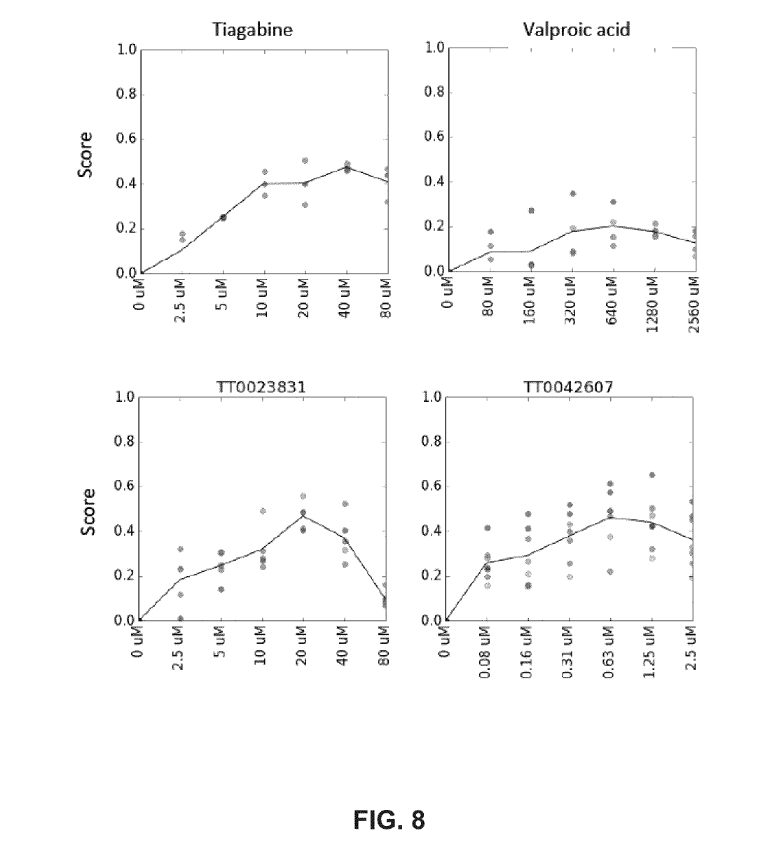

Additionally, as shown in FIG. 8, representative dose response curves for both TT0023831 and TT0042607, based on their respective suppression scores (combined measurement of VU0463271 suppression, plot distance from DMSO controls, and sedation level) at the concentrations indicated were able to outperform both tiagabine and valproic acid as suppressors of the hyperexcitability phenotype (see also FIG. 5A).

### Example 5

### Identification of Compounds that Reduce Excitability in Cultured Hippocampal Neurons

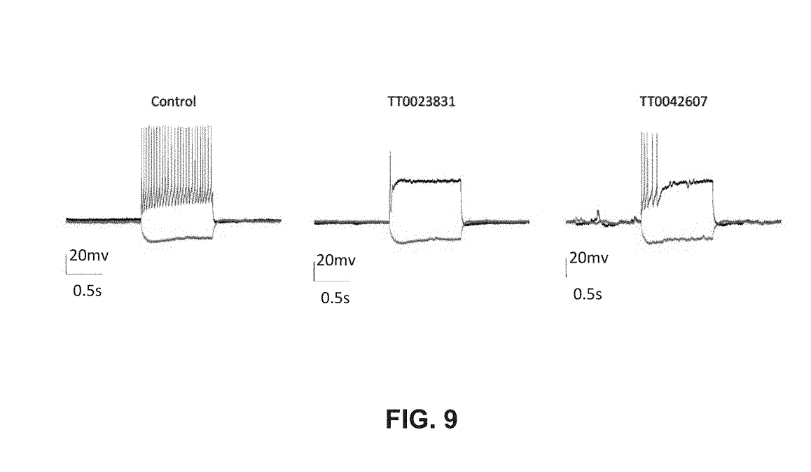

Referring to FIG. 9, representative IV curves were generated (current-voltage curves) to characterise the intrinsic excitability of TT0023831 and TT0042607 in gramicidin perforated patch clamp studies, and to investigate whether the effects of the three hit compounds identified in the above-described suppressor screens translate to rodent models. The effects of these compounds were evaluated in cultured rat hippocampal neurons using the gramicidin perforated patch technique. The GABA_{A} agonist, muscimol (1 μM), was used to measure E_{GABA} values under basal conditions (FIG. 9). Perfusion of 30 μM TT0011235 potentiated GABA_{A}-mediated currents, both muscimol-activated and endogenous post-synaptic potentials, and shifted the EGAB_{A} values (FIG. 9 and Appendix B). Although neither TT0023831 nor TT0042607 appeared to have an effect on GABA_{A}-mediated currents, both of these compounds reduced the firing rates of the cultured neurons and delayed spike initiation (FIG. 9). Analysis of action potential waveform and kinetics parameters showed that 30 μM TT0023831 increased the rheobase without changing the input resistant, thereby raising the action potential threshold, without affecting the number of channels that are open at rest (FIG. 9). Treatment with 30 μM TT0042607 slowed the upstroke and repolarization, reduced the input resistant and thus, and reduced the number of open channels.

Phenotype-based screens, as described herein, are high-content and make use of complex behaviors to identify novel modulators, which may function through various mechanisms. Thus, hits identified in the suppressor screens described herein may or may not act directly on KCC2. To test whether the three hit compounds directly alter the activity of the transporter, thallium flux assays were performed in HEK293 cells transiently transfected with human KCC2 in order to measure the influx rate of KCC2 activity (see, e.g., Appendix B). All three hit compounds identified in the suppressor screen did not accelerate the rate of 10 mM [K+]ₒ-induced thallium influx. In contrast, N-ethylmaleimide (NEM), a known positive modulator of KCC2, significantly increased the rate of KCC2-mediated flux. The KCC2 inhibitor VU0463271 significantly reduced the rate of thallium uptake. These data indicated that the three hit compounds did not increase the activity of KCC2 when over-expressed in HEK293 cells.

### Example 6

### Identification of Derivatives of TT0042607

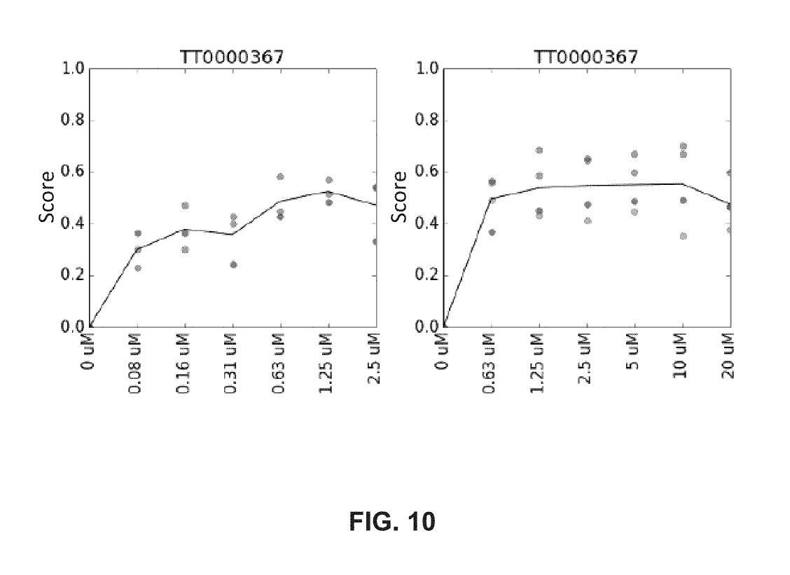

Referring to FIG. 10, representative dose response curves for a structural derivative of TT0042607 are shown (TT0000367), based on its respective suppression scores (combined measurement of VU0463271 suppression, plot distance from DMSO controls, and sedation level) at the concentrations indicated.

Structure-Activity relationship (SAR) studies were conducted using TT0042607 as the base chemical structure. Twenty structural analogs were tested and are depicted in Table 3, along with their corresponding suppression scores. Four of these twenty analogs had higher suppression scores at sub to low micromolar potency concentration ranges in vivo than TT0042607, including TT0000367. Calculation of the suppression scores of TT0000367 at the concentrations indicated in FIG. 10 demonstrate that this compound was able to outperform the other compounds tested as a suppressor of the hyperexcitability phenotype.

TABLE 3
Compound ID-Score

| | |
|---|---|
| 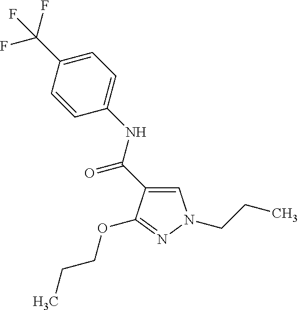 [SMILES: CCCOc1nn(CCC)cc1C(=O)Nc1ccc(C(F)(F)F)cc1] | TT0042607-44 |
| 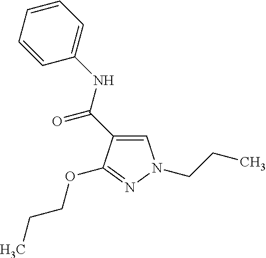 [SMILES: CCCOc1nn(CCC)cc1C(=O)Nc1ccccc1] | TT0000363-52 |
| 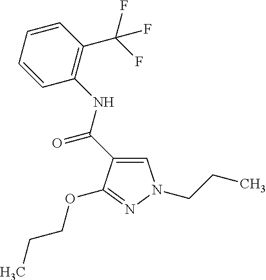 [SMILES: CCCOc1nn(CCC)cc1C(=O)Nc1ccccc1C(F)(F)F] | TT0000364-47 |
| 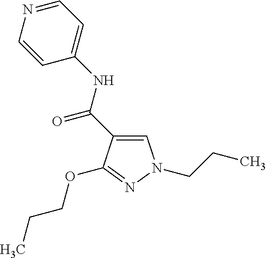 [SMILES: CCCOc1nn(CCC)cc1C(=O)Nc1ccncc1] | TT0000368-57 |
| 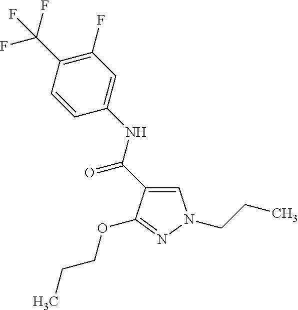 [SMILES: CCCOc1nn(CCC)cc1C(=O)Nc1ccc(C(F)(F)F)c(F)c1] | TT0000367-49 |
| 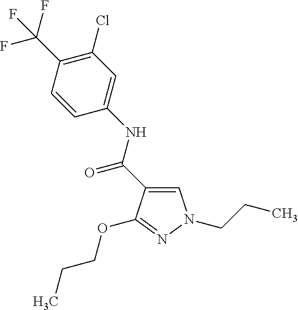 [SMILES: CCCOc1nn(CCC)cc1C(=O)Nc1ccc(C(F)(F)F)c(Cl)c1] | TT0000369-51 |
| 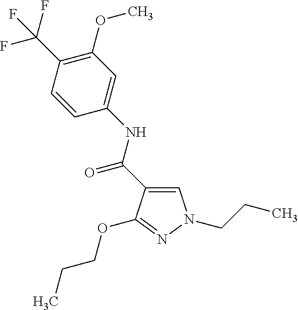 [SMILES: CCCOc1nn(CCC)cc1C(=O)Nc1ccc(C(F)(F)F)c(OC)c1] | TT0000370-50 |
| 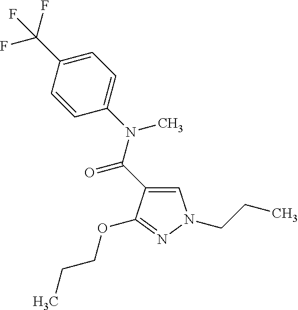 [SMILES: CCCOc1nn(CCC)cc1C(=O)N(C)c1ccc(C(F)(F)F)cc1] | TT0000371-27 |
| 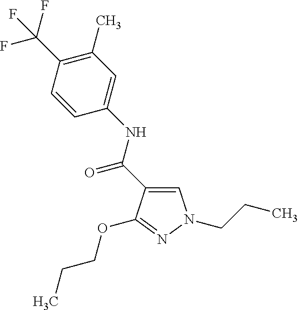 [SMILES: CCCOc1nn(CCC)cc1C(=O)Nc1ccc(C(F)(F)F)c(C)c1] | TT0000372-52 |
| 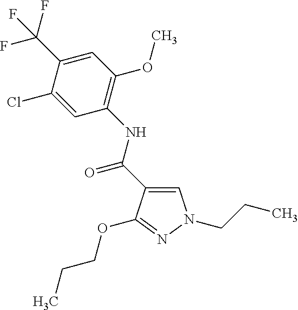 [SMILES: CCCOc1nn(CCC)cc1C(=O)Nc1cc(Cl)c(C(F)(F)F)cc1OC] | TT0000373-22 |
| 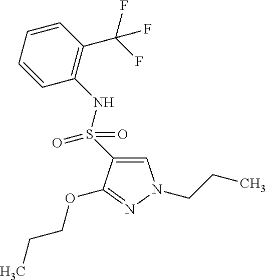 [SMILES: CCCOc1nn(CCC)cc1S(=O)(=O)Nc1ccccc1C(F)(F)F] | TT0000374-39 |
| 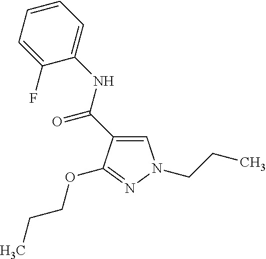 [SMILES: CCCOc1nn(CCC)cc1C(=O)Nc1ccccc1F] | TT0000347-37 |
| 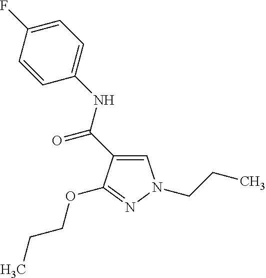 [SMILES: CCCOc1nn(CCC)cc1C(=O)Nc1ccc(F)cc1] | TT0000348-40 |
| 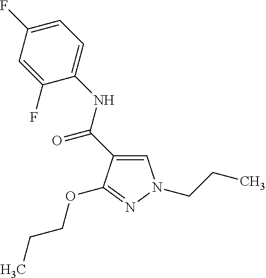 [SMILES: CCCOc1nn(CCC)cc1C(=O)Nc1ccc(F)cc1F] | TT0000349-48 |
| 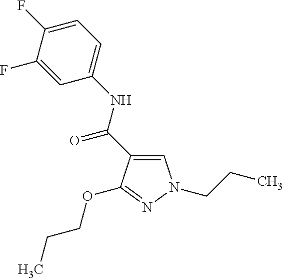 [SMILES: CCCOc1nn(CCC)cc1C(=O)Nc1ccc(F)c(F)c1] | TT0000350-40 |
| 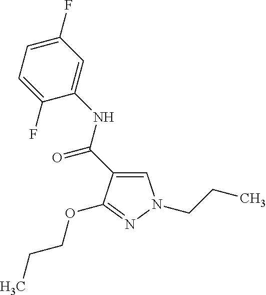 [SMILES: CCCOc1nn(CCC)cc1C(=O)Nc1cc(F)ccc1F] | TT0000351-44 |
| 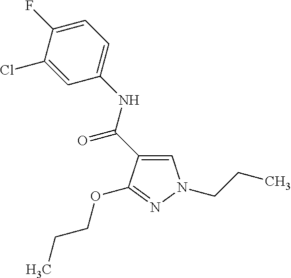 [SMILES: CCCOc1nn(CCC)cc1C(=O)Nc1ccc(F)c(Cl)c1] | TT0000352-49 |
| 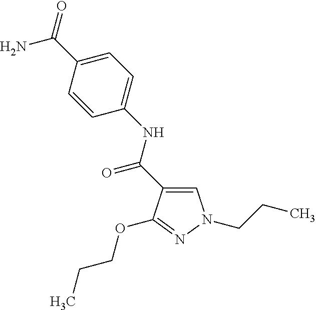 [SMILES: CCCOc1nn(CCC)cc1C(=O)Nc1ccc(C(N)=O)cc1] | TT0000353-28 |
| 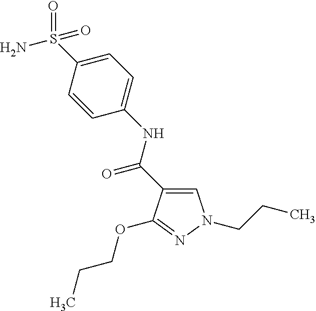 [SMILES: CCCOc1nn(CCC)cc1C(=O)Nc1ccc(S(N)(=O)=O)cc1] | TT0000354-26 |
| 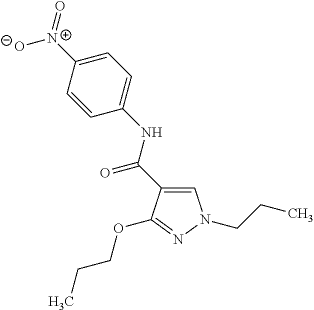 [SMILES: CCCOc1nn(CCC)cc1C(=O)Nc1ccc([N+](=O)[O-])cc1] | TT0000365-41 |
| 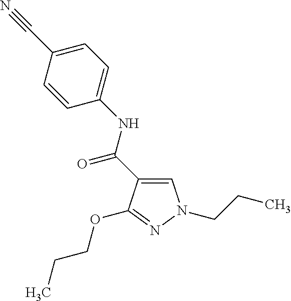 [SMILES: CCCOc1nn(CCC)cc1C(=O)Nc1ccc(C#N)cc1] | TT0000366-53 |

### Example 7

### Identification of Derivatives of TT002381

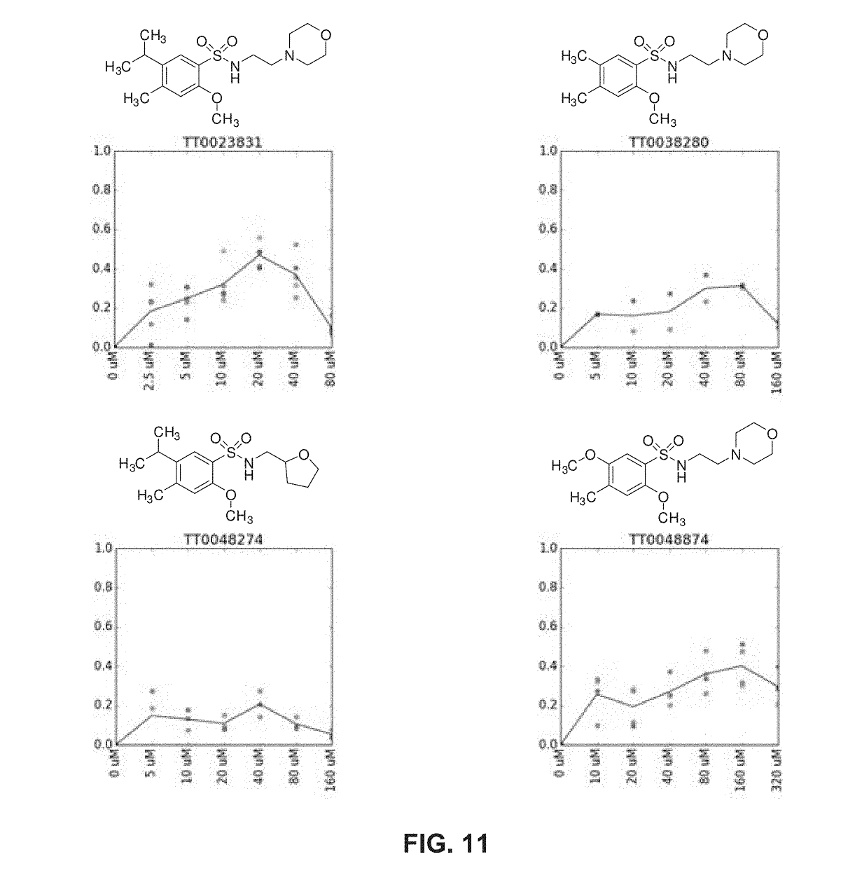

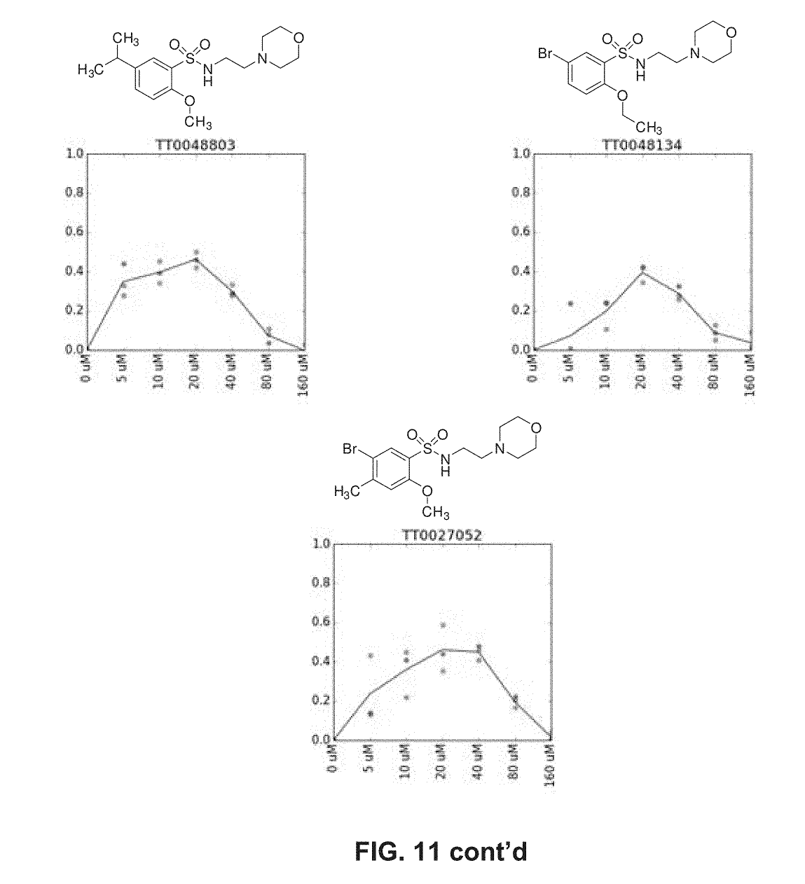

TT0023831 and six analogs were tested at a range of doses from 2.5-320 uM in a behavioral assay designed to assess the ability of compounds to suppress the effects of KCC2 inhibition. The graphs of FIG. 11 indicate the degree of suppression of the KCC2-inhibited phenotype, where a score of 1 represents complete restoration of KCC2-treated zebrafish to normal. Clinically approved antiepileptic agents typically achieve a maximal score in the range of 0.25-0.4. Table 4 summarizes the estimated EC50 values and peak effect values for each compound.

TABLE 4

| | in vivo EC50 | peak activity |
|---|---|---|
| Compound | (uM) | (uM) |
| TT0023831 | 5 | 20 |
| TT0038280 | 40 | 80 |
| TT0048274 | >160 | >160 |
| TT0048874 | 40 | 160 |
| TT0048803 | <5 | 20 |
| TT0048134 | 15 | 20 |
| TT0027052 | 5 | 20 |

### Example 8

### Animal Efficacy Studies

TT0023831 and TT0042607 were tested in mammalian models of epilepsy. The two compounds were tested in the mouse 6 Hz 44 mA model and the mouse maximal electroshock (MES) model. A range of doses was examined, and results were reported as the number of animals protected from seizures out of the number tested (Table 5).

TABLE 5

| | Dose | Rescued | Rescued |
|---|---|---|---|
| Compound | (mg/kg) | (6 Hz model) | (MES model) |
| TT0023831 | 10 | | 0/4 |
| | 30 | | 2/4 |
| | 100 | | 2/4 |
| | 50 | 0/8 | |
| | 100 | 3/8 | |
| | 200 | 7/8 | |
| TT0042607 | 10 | 0/4 | 0/4 |
| | 30 | 0/4 | 0/4 |
| | 100 | 1/4 | 2/4 |

### Example 9

### TT0042607 and its Analogs are Partial Kv7.2/7.3 (KCNQ2/3) Agonists

Eight analogs of TT0042607 were tested in a concentration-dependent manner in a CHO-Kv7.2/7.3 (KCNQ2/3) ⁸⁶Rb efflux assay. Compounds were tested at concentrations from 0.001 μM to 30 μM to generate 10-point concentration-response curve (CRC) (n=2). Retigabine and ML213 were included as control activators of Kv7.2/7.3 (KCNQ2/3). Data was normalized to ⁸⁶Rb flux elicited by application of extracellular buffer containing 70 mM KCl.

Reference Kv7.2/7.3 (KCNQ2/3) activators retigabine an ML213 elicited robust concentration-dependent ⁸⁶Rb flux with EC50 values comparable to literature values.

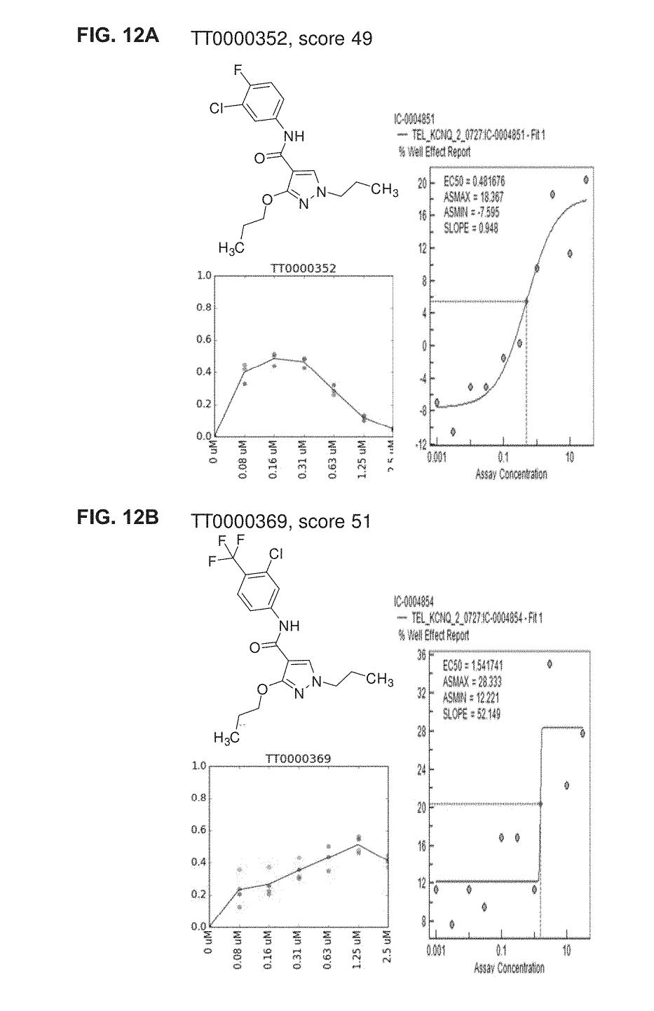

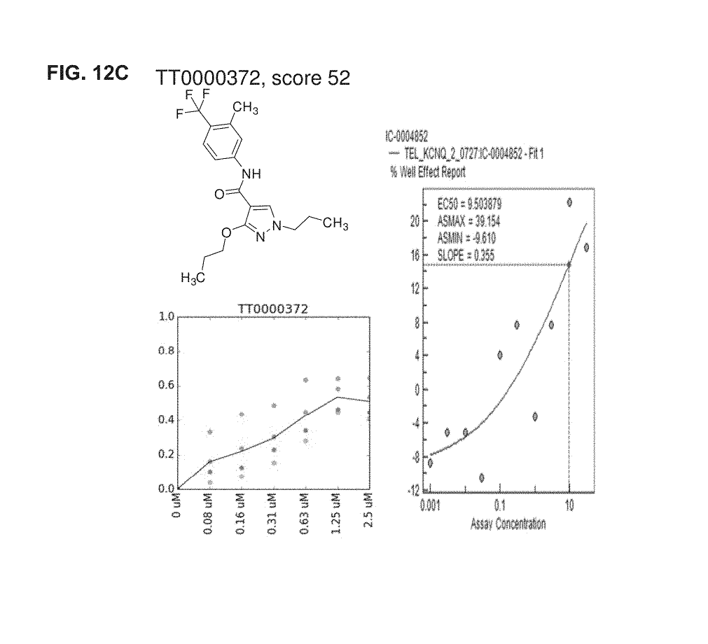

TT0042607 and its analogs exhibited partial activation of ⁸⁶Rb flux through Kv7.2/7.3 (KCNQ2/3), with maximal flux reaching ˜30% of control levels (see FIGS. 12A-12C). Data were fit with “maximum activation” set at 100% of 70 mM KCl control stimulus. Table 6 summarizes the results. Table 7 presents the suppression scores calculated for the TT0042607 analogs.

TABLE 6

| | TT0000363 | TT0000349 | TT0000351 | TT0000352 | TT0000372 | TT0000367 | TT0000369 | TT0000366 | Retigabine | ML213 |
|---|---|---|---|---|---|---|---|---|---|---|
| EC50 (μM | >30 | >30 | >30 | >30 | >30 | >30 | >30 | >30 | 0.10 | 0.02 |
| Fitted | 100 | 100 | 100 | 100 | 100 | 100 | 100 | 100 | 82 | 98 |
| Max % | | | | | | | | | | |
| Max Flux % | 15 | 33 | 33 | 33 | 31 | 21 | 35 | 28 | 89 | 109 |
| Conc μM @ | 0.001 | 10 | 10 | 3 | 10 | 0.01 | 3 | 3 | 10 | 0.3 |
| Max Flux | | | | | | | | | | |

TABLE 7

| | TT0000363 | TT0000349 | TT0000351 | TT0000352 | TT0000372 | TT0000367 | TT0000369 | TT0000366 |
|---|---|---|---|---|---|---|---|---|
| EC50 (μM, raw) | >30 | 0.54 | >30 | 0.48 | 9.5 | >30 | 1.54 | 0.0054 |
| | >30 | >30 | >10 | 0.14 | 1.6 | >30 | 2.63 | >30 |
| Suppression Score | 52 | 48 | 44 | 49 | 52 | 49 | 51 | 53 |

### Example 10

### TT0023831 is a Sodium Channel Antagonist

TT002381 was tested in a concentration-dependent manner in a sodium channel FLIPR assay. TT002381 was tested to a maximum concentration of 100 μM to generate a 10-point concentration-response curve (CRC). Tetracaine and TTX were included as control inhibitors.

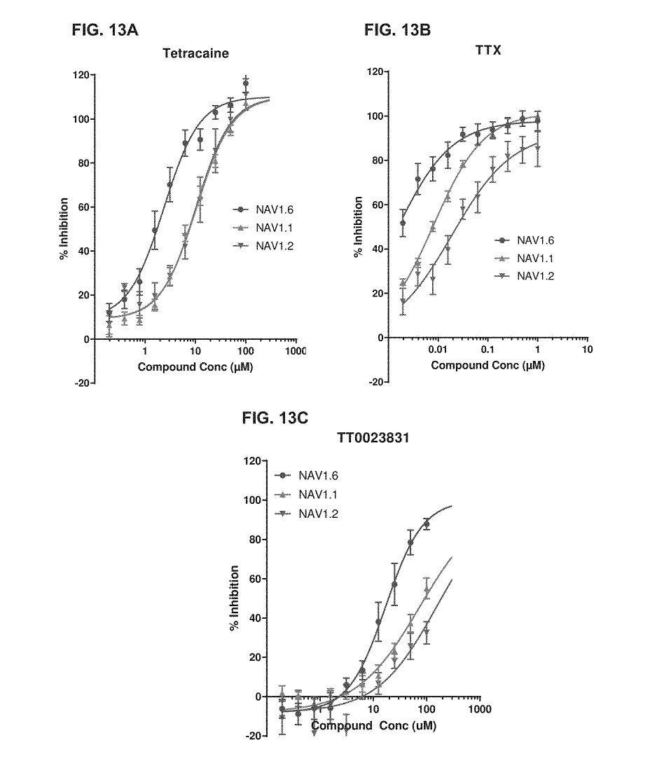

As depicted in FIG. 13, tetracaine and TTX elicited concentration-dependent inhibition of Nav1.1, 1.2, and 1.6. Similarly, TT002381 elicited concentration-dependent inhibition of Nav1.1, 1.2, and 1.6.

### Materials and Methods

### Aquaculture and Chemical Treatment

A large number of fertilized zebrafish eggs (up to 20,000 embryos per day) were collected from group matings of wild type Ekkwill/Singapore zebrafish. Embryos were raised in hatching jars at 28° C. on a 14/10 hour light/dark cycle until 3 days post fertilization (dpf), then transferred to an incubator under the same conditions until 7 dpf. Groups of eight larvae (7 dpf) were distributed into the wells of a clear flat bottom square well 96-well plates filled with E3 medium (300 μl). Larvae were then incubated at 25° C. on the bench top for one-hour chemical treatment and subsequent experiments.

### Suppressor Screens and Retesting

A library containing 37,000 uncharacterized small molecules was purchased from Life Chemicals and dissolved in DMSO at a stock concentration of 10 mM. This collection was computationally enriched for SP3 centers, chemical diversity and drug likeliness. All compounds were diluted in E3 buffer and screened at either 40 μM (VU0463271 primary suppressor screen) or 10 μM (VU0240551 primary suppressor screen) final concentration and <1% DMSO. Negative controls were treated with an equal volume of DMSO. Stock solutions were added directly to zebrafish in the wells of a 96-well plate, mixed and allowed to incubate for 50 min at the bench and 10 min dark adaptation before a first behavioral evaluation in the behavioral battery of assays. After testing (approximately 15 min), all 96-well plates were incubated with either 2.5 μM VU0463271 or 5 μM VU0240551 for 30 min plus 10 min dark adaptation prior to their second evaluation in the assay battery. Within each screening plate, the first column (8 wells) was treated with DMSO only and the last column with an inhibitor only (no library compound). Reordered hit compounds and reference compounds were dissolved in DMSO to a stock concentration of 60 mM and ×300 stock solution was added directly to the wells. Reference and reordered compounds were retested by generating 6-point dose response curves at the indicated concentrations and with the same incubation times as in the screen. Each concentration of the selected compound, including DMSO or KCC2 inhibitor control was added to an entire row (12 wells). Retesting was performed in triplicate.

Finally, it should be noted that there are alternative ways of implementing the subject matter provided by the present disclosure. Accordingly, the disclosed embodiments are to be considered as illustrative and not restrictive, and the present disclosure is not to be limited to the details given herein, but may be modified within the scope and equivalents of the appended claims.

What is claimed is:

1. A pharmaceutical composition comprising a compound of Formula 1: 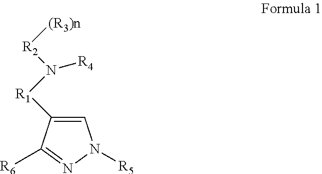
or a pharmaceutically acceptable salt, hydrate, solvate, or N-oxide thereof, wherein:
R₁ is selected from carbonyl and sulfonyl;
R₂ is selected from phenyl, pyridine, and thiazole;
n is 0, 1, 2, 3, or 4;
each R₃ is independently selected from H, halogen, C₁-C₄ alkyl, C₁-C₄ alkoxy, C₁-C₄ haloalkyl, amide, acetamide, sulfonamide, sulfone, nitro, nitrile, and alkane nitrate;
R₄ is selected from H and C₁-C₄ alkyl;
R₅ is C₁-C₆ alkyl; and
R₆ is C₁-C₆ alkoxy; and
a pharmaceutically acceptable vehicle.

2. The pharmaceutical composition of claim 1, wherein each R₃ is independently selected from methyl, methoxy, trifluoromethyl, fluoride, and chloride.

3. The pharmaceutical composition of claim 1, wherein R₄ is selected from H and methyl.

4. The pharmaceutical composition of claim 1, wherein n is 1.

5. The pharmaceutical composition of claim 1, wherein R₅ is propyl.

6. The pharmaceutical composition of claim 1, wherein R₆ is propoxy.

7. The pharmaceutical composition of claim 1, wherein the compound of Formula 1 is selected from:

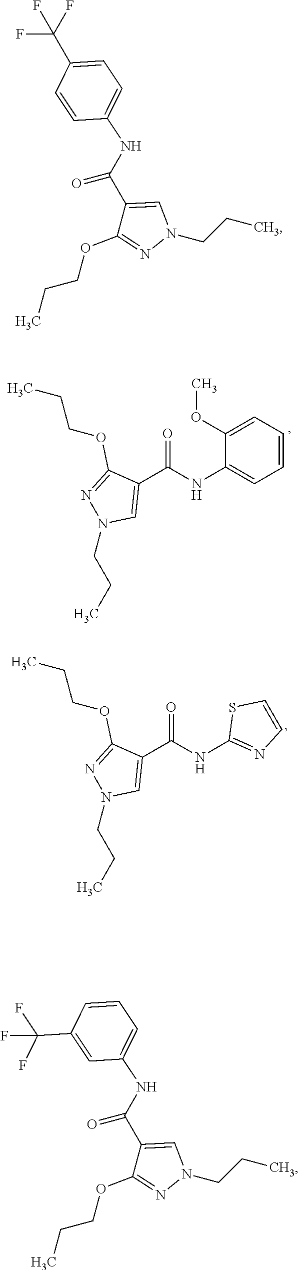 [SMILES: CCCOc1nn(CCC)cc1C(=O)Nc1ccc(C(F)(F)F)cc1 ; CCCOc1nn(CCC)cc1C(=O)Nc1ccccc1OC ; CCCOc1nn(CCC)cc1C(=O)Nc1nccs1 ; CCCOc1nn(CCC)cc1C(=O)Nc1cccc(C(F)(F)F)c1]

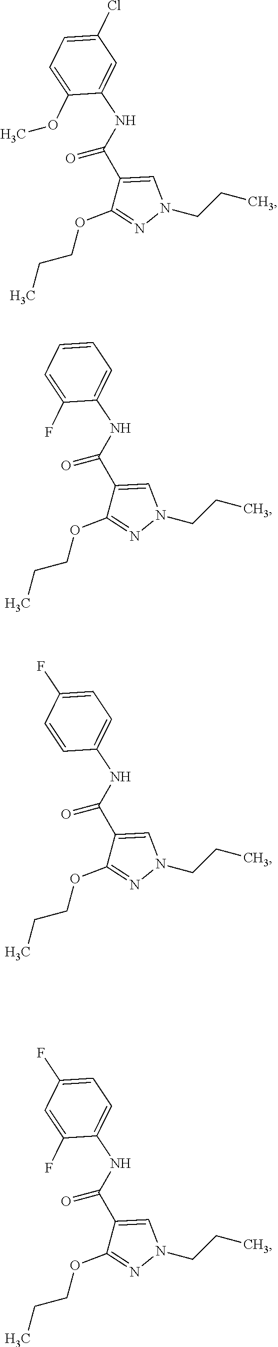 [SMILES: CCCOc1nn(CCC)cc1C(=O)Nc1cc(Cl)ccc1OC ; CCCOc1nn(CCC)cc1C(=O)Nc1ccccc1F ; CCCOc1nn(CCC)cc1C(=O)Nc1ccc(F)cc1 ; CCCOc1nn(CCC)cc1C(=O)Nc1ccc(F)cc1F]

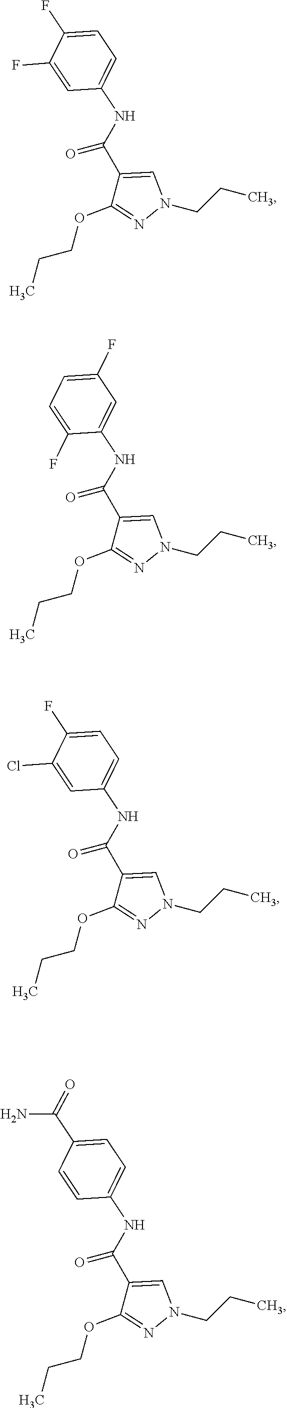 [SMILES: CCCOc1nn(CCC)cc1C(=O)Nc1ccc(F)c(F)c1 ; CCCOc1nn(CCC)cc1C(=O)Nc1cc(F)ccc1F ; CCCOc1nn(CCC)cc1C(=O)Nc1ccc(F)c(Cl)c1 ; CCCOc1nn(CCC)cc1C(=O)Nc1ccc(C(N)=O)cc1]

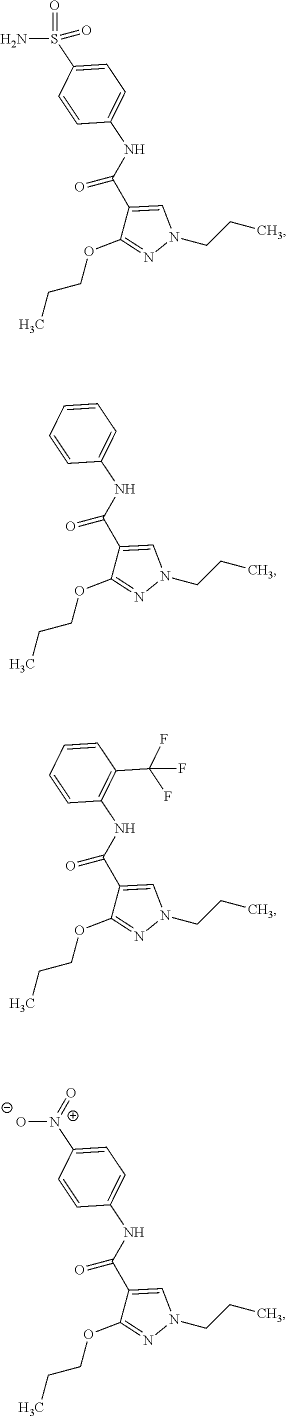 [SMILES: CCCOc1nn(CCC)cc1C(=O)Nc1ccc(S(N)(=O)=O)cc1 ; CCCOc1nn(CCC)cc1C(=O)Nc1ccccc1 ; CCCOc1nn(CCC)cc1C(=O)Nc1ccccc1C(F)(F)F ; CCCOc1nn(CCC)cc1C(=O)Nc1ccc([N+](=O)[O-])cc1]

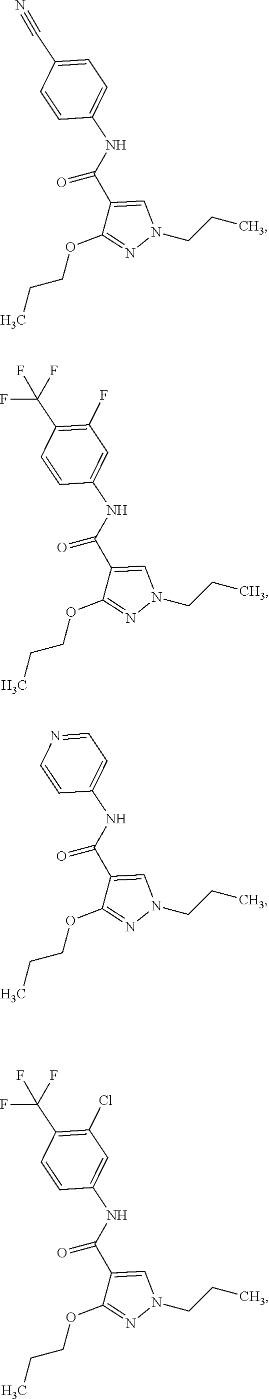 [SMILES: CCCOc1nn(CCC)cc1C(=O)Nc1ccc(C#N)cc1 ; CCCOc1nn(CCC)cc1C(=O)Nc1ccc(C(F)(F)F)c(F)c1 ; CCCOc1nn(CCC)cc1C(=O)Nc1ccncc1 ; CCCOc1nn(CCC)cc1C(=O)Nc1ccc(C(F)(F)F)c(Cl)c1]

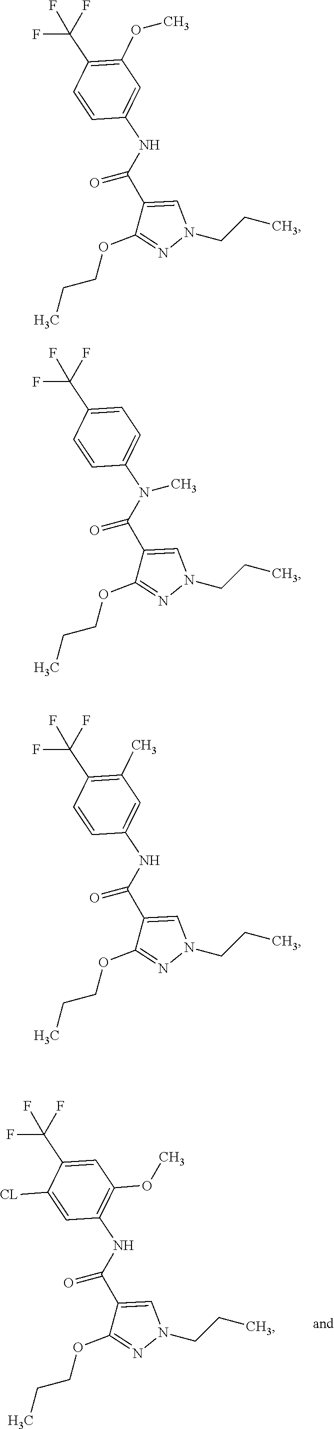

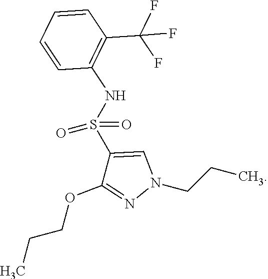 [SMILES: CCCOc1nn(CCC)cc1S(=O)(=O)Nc1ccccc1C(F)(F)F]

8. The pharmaceutical composition of claim 1, wherein the pharmaceutical composition is formulated for oral administration.

9. The pharmaceutical composition of claim 8, wherein the pharmaceutical composition comprises 2.5 mg to 1000 mg of the compound of Formula 1.

10. The pharmaceutical composition of claim 8, wherein the pharmaceutical composition comprises an amount of the compound of Formula 1 selected from 2.5 mg, 5 mg, 10 mg, 25 mg, 50 mg, 100 mg, 250 mg, 500 mg, and 1000 mg.

11. A method for treating a disease selected from epilepsy, neuropathic pain, spinal cord injury spasticity, morphine-induced hyperalgesia, schizophrenia, autism spectrum disorders and combinations thereof, the method comprising administering a pharmaceutical compound of claim 1 to a patient.

12. The method of claim 11, wherein the pharmaceutical compound is administered in a dose of 5 mg to 2.5 g per day of the compound of Formula 1.

13. A pharmaceutical composition comprising a compound of Formula 2: 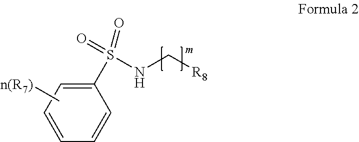
or a pharmaceutically acceptable salt, hydrate, solvate, or N-oxide thereof, wherein:
n is 1, 2, 3, or 4;
each R₇ is independently selected from C₁-C₄ straight chain or branched alkyl, C₁-C₄ alkoxy, and halogen;
m is 1, 2, 3, or 4; and
R₈ is selected from tetrahydrofuran and a morpholine moiety; and
a pharmaceutically acceptable vehicle.

14. The pharmaceutical composition of claim 13, wherein n is selected from 2 and 3.

15. The pharmaceutical composition of claim 13, wherein each R₇ is independently selected from methyl, methoxy, isopropyl, and bromide.

16. The pharmaceutical composition of claim 13, wherein m is selected from 1 and 2.

17. The pharmaceutical composition of claim 13, wherein the compound of Formula 2 is selected from:

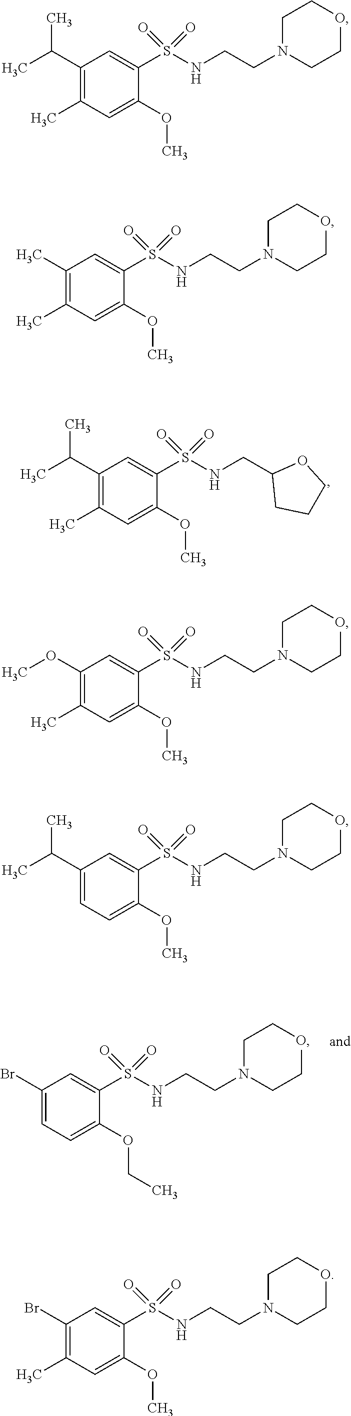

18. The pharmaceutical composition of claim 13, wherein the pharmaceutical composition is formulated for oral administration.

19. The pharmaceutical composition of claim 18, wherein the pharmaceutical composition comprises 2.5 mg to 1000 mg of the compound of Formula 2.

20. The pharmaceutical composition of claim 18, wherein the pharmaceutical composition comprises an amount of the compound of Formula 2 selected from 2.5 mg, 5 mg, 10 mg, 25 mg, 50 mg, 100 mg, 250 mg, 500 mg, and 1000 mg.

21. A method for treating a disease and/or condition selected from epilepsy, neuropathic pain, spinal cord injury spasticity, morphine-induced hyperalgesia, schizophrenia, autism spectrum disorders and combinations thereof, the method comprising administering a pharmaceutical compound of claim 13 to a patient.

22. The method of claim 21, wherein the pharmaceutical compound is administered in a dose of 5 mg to 2.5 g per day of the compound of Formula 2.
